(19) United States
(12) Patent Application Publication
(10) Pub. No.: US 2025/0230419 A1
(43) Pub. Date: Jul. 17, 2025

(54) METHODS OF PURIFYING AN ENVELOPED VIRUS

(71) Applicant: CSL Behring LLC, King of Prussia (PA)

(72) Inventors: Tobias BRANDT, Marburg (DE); Holger LAUX, Marburg (DE); Jessica Elisabeth VOGEL, Marburg (DE)

(21) Appl. No.: 18/851,832

(22) PCT Filed: Mar. 30, 2023
(86) PCT No.: PCT/IB2023/053159

§ 371 Date: Sep. 27, 2024

Related U.S. Application Data
(60) Provisional application No. 63/362,163, filed on Mar. 30, 2022.

(51) Int. Cl. C12N 7/00; B01D 15/20; B01D 15/36; B01D 15/42; B01D 61/14; C12N 9/22

(52) U.S. Cl. CPC C12N 7/00; B01D 15/203; B01D 15/363; B01D 15/426; B01D 61/145; C12N 9/22; C12Y 301/30002; B01D 2315/10; B01D 2315/16; C12N 2740/10021; C12N 2740/10051

(57) ABSTRACT

The present disclosure relates generally to the manufacturing of gene therapy products, and specifically to methods of purifying an enveloped virus from a cell culture fluid, comprising an endonuclease and/or anion exchange chromatography.

### RELATED APPLICATION DATA

The present application claims priority from U.S. provisional application No. 63/362,163 entitled ‘Methods of purifying an enveloped virus’ filed 30 Mar. 2022. The entire contents of which is hereby incorporated by reference.

### FIELD

The present disclosure relates generally to the manufacturing of gene therapy products, and specifically to methods of purifying an enveloped virus from a cell culture fluid, comprising an endonuclease and/or anion exchange chromatography.

### BACKGROUND

Retroviruses, e.g., lentiviruses are one of the most studied viral vectors for gene therapy. Retroviruses in general are RNA-based viruses which integrate their genetic information into the target cell chromosomes permanently. The advantages of retroviruses include long-term transgene expression in target cells, a low immunogenic potential, and the ability to transduce into dividing and non-dividing cells.

Lentiviruses are genetically engineered and usually based on human immunodeficiency virus 1 (HIV-1). To increase safety, modern vectors contain only those HIV genes which are necessary for infection and gene delivery, but the genes necessary for replication and virulence factors have been removed. Often, the envelope protein of HIV-1 is exchanged with that of another virus to allow infection of a wide range of target cells, e.g., VSV-G protein from Vesicular stomatitis Indiana virus (VSV).

To produce lentiviruses, cells such as human embryonic kidney cells HEK 293T are transfected with 3-4 plasmids. These include the transfer plasmid with the gene of interest and several packaging plasmids encoding, vesicular stomatitis G protein (VSV-G), and essential viral proteins responsible for gene integration or self-assembly. These plasmids can be transiently transfected into the cells, or a producer cell line is created with stable integration of the plasmids with inducible promoters, in which lentivirus production can be induced.

Once the virus production has been induced, the release of the virus occurs by budding after successful assembly within the cells.

The lentivirus is harvested from the producer cells and subsequently purified and concentrated in the downstream process. However, purification of lentiviruses at commercial scale is difficult. Limiting obstacles for the purification of this type of virus are the impurities that are produced with large scale cell culture and the instability of certain membrane glycoproteins when exposed to some purification conditions. Thus, there is a need in the art for an efficient process for purifying lentiviruses, e.g., for gene therapy.

### SUMMARY

In work leading up to the present invention, the inventors sought to produce a method for producing enveloped viruses, e.g., for gene therapy, at commercial scale and suitable for regulatory requirements. The inventors also sought to develop a method that could be performed in-line or in a continuous or semi-continuous manner.

In some examples, the downstream process for purifying and concentrating viral vector produced by the inventors includes a harvest filtration step to remove cellular debris and components and a purification step to reduce overall volume and to separate viral vector from host cell DNA, proteins, and media components. The downstream process for purifying and concentrating viral vector produced by the inventors can additionally include an ultrafiltration/diafiltration step to concentrate the viral vector into a final formulation buffer. In some examples, the downstream step further includes a sterile filtration step for removal of microorganisms from the final product.

In developing this method, the inventors determined that the nucleic acids in cell culture fluid in which the virus was produced (e.g., the supernatant from stable cell lines) were causing fouling of membranes, resins, and filters used in the downstream process (e.g., in purification by anion exchange chromatography and in the sterile filtration step). To address this problem, the inventors added an endonuclease to the cell culture fluid. The inventors identified that they could add the endonuclease prior to purification, which would improve purification as well as other downstream steps such as sterile filtration. In one example, the inventors found that they could add the endonuclease immediately before harvest filtration and then proceed to purification without requiring a separate incubation with the endonuclease. The addition of endonuclease facilitated longer harvesting and higher vector yields without clogging of chromatography columns and/or sterile filtration filters, for virus produced by both adherent cells and suspension cells. The inventors additionally identified that it was desirable to increase the salt concentration in a cell culture fluid prior to anion exchange chromatography to reduce impurity binding, and thus to facilitate virus binding to the anion exchanger and contaminants flowing through. However, increasing the salt concentration is complicated by several factors. For one, it is undesirable to add substantial volumes to the cell culture fluid prior to anion exchange, and therefore a high-concentration salt solution is preferred. However, hypertonic conditions destabilize viral particles. Additionally, manual mixing at commercial scale is cumbersome and time-consuming and not amenable to in-line processing. When mixing is performed in a container, if the salt is not adequately mixed, the denser high-salt solution settles to the bottom of the container, and higher local salt concentrations can cause virus to elute during loading of the chromatography unit. To address these problems, the inventors add a high concentration salt solution to the cell culture fluid by flowing two fluid streams together immediately before chromatography. The high concentration salt solution and the cell culture fluid are flowed together to cause them to mix as they are being loaded onto an anion exchanger, or immediately before loading onto the anion exchanger, or within the anion exchanger. This facilitates mixing of the solutions without adding substantial volume and without destabilizing the virus. It also allows efficient mixing without requiring a time-consuming mixing step, whether at lab scale or commercial scale.

The findings by the inventors have provided methods for purifying enveloped viruses.

In one example, the disclosure provides a method of purifying an enveloped virus from a cell culture fluid, comprising contacting cell culture fluid with an endonuclease prior to harvest filtration.

The disclosure additionally provides a method of purifying an enveloped virus from a cell culture fluid or a filtered cell culture fluid, comprising contacting the cell culture fluid or the filtered cell culture fluid with an endonuclease prior to purifying the virus.

In exemplary forms of the disclosure, the cell culture fluid or harvested cell culture fluid is from stable producer cells, i.e., cells having stably incorporated therein the genetic material required to produce the lentivirus. Such cells are distinguished from cells having the genetic elements transiently incorporated therein.

An exemplary enveloped virus is a retrovirus. For example, the retrovirus is a lentivirus. For example, the lentivirus is HIV or a derivative thereof.

An exemplary nuclease is a non-specific endonuclease and degrades both DNA and RNA without sequence specificity. In one example, the endonuclease is a non-specific endonuclease and degrades DNA without sequence specificity.

Suitable endonucleases are known in the art and include those from Serratia marcescens, Anabaena sp., Saccharomyces cerevisiae, Bos Taurus, Syncephalostrum racemosum and/or Borrelia burgdorferi. For example, the endonuclease is a Serratia nuclease, NucA, Nuc1, endonuclease G, DNase I, or micrococcal nuclease.

The endonuclease can be added at any concentration suitable to prevent fouling of a membrane or anion exchanger following endonuclease treatment. Suitable concentrations are described herein. In one example, the endonuclease is added to the cell culture fluid at a concentration of 0.001 to 100 units/mL of cell culture fluid. In one example, the endonuclease is added to the cell culture fluid at a concentration of 0.01 to units/mL of cell culture fluid. In one example, the endonuclease is added to the cell culture fluid at a concentration of 0.1 to 1 unit/mL of cell culture fluid. For example, the endonuclease is added to the cell culture fluid at a concentration of 0.3 units/mL of cell culture fluid.

The endonuclease is added before purification, e.g., before anion exchange. The disclosure encompasses incubating the endonuclease and the cell culture fluid prior to anion exchange. In some examples, the endonuclease and the cell culture fluid are incubated for less than about 2 hours or less than about 1 hour before anion exchange.

The inventors have shown that in certain examples there is no need to include a separate incubation for the endonuclease and the cell culture fluid. For example, the endonuclease can be added to the cell culture fluid prior to harvest filtration and the cell culture fluid can then immediately proceed to harvest filtration, since the processing time of the harvest filtration process itself before purification, e.g., anion exchange, is sufficient to prevent fouling of the anion exchanger. However, the present disclosure also encompasses incubating the endonuclease and the cell culture fluid prior to harvest filtration. In some examples, the endonuclease and the cell culture fluid are incubated for less than about 2 hours or less than about 1 hour before harvest filtration.

In some examples, purification is performed less than about 30 hours after contacting the cell culture fluid or the filtered cell culture fluid with the endonuclease, e.g., less than about 22 hours after contacting the cell culture fluid or the filtered cell culture fluid with the endonuclease, such as less than about 6 hours after contacting the cell culture fluid or the filtered cell culture fluid with the endonuclease, for example, less than about 4 hours after contacting the cell culture fluid or the filtered cell culture fluid with the endonuclease, e.g., less than about 2 hours after contacting the cell culture fluid or the filtered cell culture fluid with the endonuclease, e.g., less than about 1 hour after contacting the cell culture fluid or the filtered cell culture fluid with the endonuclease, or less than about e.g., 30 minutes after contacting the cell culture fluid or the filtered cell culture fluid with the endonuclease.

In one example, the method further comprises performing harvest filtration immediately after contacting the cell culture fluid with the endonuclease to produce a filtered cell culture fluid.

In some examples, the endonuclease is contacted to the cell culture fluid or the filtered cell culture fluid at a concentration of about 0.3 units/mL of cell culture fluid or filtered cell culture fluid, and purification is performed between about 1 and about 2 hours after contact.

In examples of the disclosure the endonuclease is contacted to the cell culture fluid or the filtered cell culture fluid at a concentration of between about 0.01 and 0.3 units/mL of cell culture fluid or filtered cell culture fluid, and purification is performed at greater than about 2 hours after contact.

In additional examples of the disclosure, the endonuclease is contacted to the cell culture fluid or the filtered cell culture fluid at a concentration of between about 0.3 and 10 units/mL of cell culture fluid or filtered cell culture fluid, and purification is performed at less than about 1 hour after contact.

In a further example of the disclosure the endonuclease is contacted to the cell culture fluid or the filtered cell culture fluid at a concentration of about 0.3 units/mL of cell culture fluid or filtered cell culture fluid, and purification is performed at less than about 1 hour after contact.

In one example, the method further comprises contacting the cell culture fluid or the filtered cell culture fluid with a magnesium salt prior to purifying the virus. In one example, the magnesium salt is added in an amount to achieve a target magnesium concentration of less than 10 mM Mg²⁺. For example, 0.001 mM to 10 mM, or 0.1 mM to 10 mM, or 1 mM to 10 mM. In one example, the magnesium salt is added in an amount to achieve a target magnesium concentration of less than 10 mM, such as 9 mM, or 8 mM, or 7 mM or 6 mM. In one example, the magnesium salt is added in an amount to achieve a target magnesium concentration of less than 5 mM. For example, 5 mM, or 4 mM, or 3 mM, or 2 mM, or 1 mM. In one example, the magnesium salt is added in an amount to achieve a target magnesium concentration of about 2 mM.

Therefore to achieve the target magnesium concentration, the ratio of filtered cell culture fluid or cell culture fluid to the magnesium salt solution is dependent on the concentration of the magnesium salt solution. In one example, the magnesium salt solution is at a concentration of less than 500 mM, for example, 50 mM to 500 mM or 100 mM to 400 mM, or 150 mM to 300 mM. For example, the magnesium salt solution is at a concentration of 200 mM.

In one example, the magnesium salt is magnesium chloride. Other suitable forms of magnesium salts suitable for use in the present disclosure will be apparent to the skilled person and/or described herein.

In one example, the method further comprises subjecting the filtered cell culture fluid to anion exchange chromatography. For example, the filtered cell culture fluid is subjected to anion exchange chromatography immediately after harvest filtration.

As discussed above, the inventors have shown that contacting a cell culture fluid or a filtered cell culture fluid with a high concentration salt solution to form a salt-spiked cell culture fluid prior to or during loading on to the anion exchange chromatography column improves purification by anion exchange, e.g., by preventing impurity binding to the anion exchanger, thus enhancing binding of the virus and permitting contaminants to flow through.

Thus in some examples, the filtered cell culture fluid is contacted with a high concentration salt solution to form a salt-spiked cell culture fluid prior to or during loading on to the anion exchange chromatography column. Such a form of mixing facilitates in-line processing. For example, a cell culture fluid can be contacted with an endonuclease and then filtered and then as it is directly loaded onto an anion exchange column, mixed with a high salt concentration solution.

The present disclosure additionally provides a method of purifying an enveloped virus from a cell culture fluid using anion exchange chromatography, wherein the cell culture fluid is contacted with a high concentration salt solution to form a salt-spiked cell culture fluid prior to or during loading on to the anion exchange chromatography column.

The anion exchanger can be a resin-based anion exchanger, an anion exchange membrane adsorber, or any other format of anion exchanger with a positively charged substrate for capturing negatively charged particles. In an exemplified form of the disclosure, the anion exchanger is an anion exchange membrane adsorber.

In one example, the high concentration salt solution and the filtered cell culture fluid or the cell culture fluid are mixed, e.g., in-line during loading on to the anion exchange chromatography column. For example, during loading onto an anion exchange column, the fluids are separately added to the column at the same time. In some examples, the fluids may contact each other prior to entering the anion exchange column, after entering the anion exchange column, or at the same time as entering the anion exchange column.

In one example, the mixed filtered cell culture fluid or the cell culture fluid and the high concentration salt solution have a target salt concentration of about 300 mM to 500 mM salt for loading onto the anion exchange chromatography column. For example, the target salt concentration is 400 mM salt.

Therefore to achieve the target salt concentration, the ratio of filtered cell culture fluid or cell culture fluid to high concentration salt solution is dependent on the concentration of the salt solution. In one example, the high concentration salt solution is at a concentration of at least 1M, for example, 1M to 10M or 2M to 9M or 3M to 8M or 4M to 6M. For example, the high concentration salt solution is at a concentration of 5M.

In one example, the filtered cell culture fluid or the cell culture fluid and the high concentration salt solution are mixed at a ratio of about 70-99% (v/v) filtered cell culture fluid or cell culture fluid and about 1-30% (v/v) high concentration salt solution. In one example, the high concentration salt solution is provided at a concentration of 5M, and the filtered cell culture fluid or the cell culture fluid and the high concentration salt solution are mixed at a ratio of 90-95% (v/v) filtered cell culture fluid or cell culture fluid and 5-10% (v/v) high concentration salt solution. For example, the filtered cell culture fluid or the cell culture fluid and the high concentration salt solution are mixed at a ratio of 95% (v/v) filtered cell culture fluid or cell culture fluid and 5% (v/v) high concentration salt solution. For example, the filtered cell culture fluid or the cell culture fluid and the high concentration salt solution are mixed at a ratio of 94% (v/v) filtered cell culture fluid or cell culture fluid and 6% (v/v) high concentration salt solution. For example, the filtered cell culture fluid or the cell culture fluid and the high concentration salt solution are mixed at a ratio of 93% (v/v) filtered cell culture fluid or cell culture fluid and 7% (v/v) high concentration salt solution.

In another example, the high concentration salt solution is provided at a concentration of 1M, and the filtered cell culture fluid or the cell culture fluid and the high concentration salt solution are mixed at a ratio of about 70% (v/v) filtered cell culture fluid or cell culture fluid and about 30% (v/v) high concentration salt solution.

In another example, the high concentration salt solution is provided at a concentration of 2M, and the filtered cell culture fluid or the cell culture fluid and the high concentration salt solution are mixed at a ratio of about 85% (v/v) filtered cell culture fluid or cell culture fluid and about 15% (v/v) high concentration salt solution.

In another example, the high concentration salt solution is provided at a concentration of 10M, and the filtered cell culture fluid or the cell culture fluid and the high concentration salt solution are mixed at a ratio of about 97% (v/v) filtered cell culture fluid or cell culture fluid and about 3% (v/v) high concentration salt solution.

In one example, the flow rate of the fluid stream containing the filtered cell culture fluid or the cell culture fluid is higher than the flow rate of the fluid stream containing the high concentration salt solution. In another example, the flow rate of the fluid stream containing the filtered cell culture fluid or the cell culture fluid is lower than the flow rate of the fluid stream containing the high concentration salt solution. In another example, the flow rate of the fluid stream containing the filtered cell culture fluid or the cell culture fluid is the same as the flow rate of the fluid stream containing the high concentration salt solution.

In one example, the mixed filtered cell culture fluid or the cell culture fluid and the high concentration salt solution have a target conductivity of 35 to 45 mS/cm at 25° C. In one example, the mixed filtered cell culture fluid or the cell culture fluid and the high concentration salt solution have a target conductivity of 36 to 44 mS/cm at 25° C. For example, a target conductivity of about 40+4 mS/cm at 25° C.

In one example, the high concentration salt solution comprises a monovalent and/or a divalent salt. For example, the high concentration salt solution comprises a monovalent salt, i.e., as the only salt. In one example, the monovalent salt is sodium chloride.

In one example, the high concentration salt solution is sodium chloride at a concentration of at least about 1M, e.g., at a concentration of about 5M.

In one example, the salt-spiked cell culture fluid has a salt concentration of 300 mM to 500 mM, e.g., about 400 mM.

In one example, the method further comprises washing the anion exchange chromatography column with one or more wash steps. For example, the method comprises a first wash step with a solution comprising 90-95% (v/v) of a buffer and 5-10% (v/v) of the high concentration salt solution. For example, the solution comprises 10-100 mM Tris, 150 mM NaCl, pH 7.0-9.0. In one example, the solution comprises 50 mM Tris, 150 mM NaCl, pH 8. In another example, the solution comprises 5-50 mM histidine, 150 mM NaCl, pH 5.5-7.4. In another example, the solution comprises 10 mM histidine, 150 mM NaCl, pH 7. In another example, the solution comprises 5-50 mM HEPES, 150 mM NaCl, pH 6.8-8.2. In another example, the solution comprises 10 mM HEPES, 150 mM NaCl, pH 7.5.

In one example, the method comprises a second wash step with a second solution. For example, the second solution comprises 10-100 mM Tris, 750 mM NaCl, pH 7.0-9.0. In one example, the second solution comprises 50 mM Tris, 750 mM NaCl, pH 8. In another example, the second solution comprises 5-50 mM histidine, 750 mM NaCl, pH 5.5-7.4. In another example, the second solution comprises 10 mM histidine, 750 mM NaCl, pH 7. In another example, the second solution comprises 5-50 mM HEPES, 750 mM NaCl, pH 6.8-8.2. In another example, the second solution comprises 10 mM HEPES, 750 mM NaCl, pH 7.5.

In one example, the method further comprises eluting bound virus from the anion exchange chromatography column with an elution solution. For example, the elution solution comprises 10-100 mM Tris, 1 M to 2 M NaCl, pH 7.0-9.0. In one example, the elution solution comprises 50 mM Tris, 1.2 M NaCl, pH 8. In another example, the elution solution comprises 50 mM Tris, 1.5 M NaCl, pH 8. In another example, the elution solution comprises 5-50 mM histidine, 1 M to 2 M NaCl, pH 5.5-7.4. In another example, the elution solution comprises 10 mM histidine, 1.2 M NaCl, pH 7. In another example, the elution solution comprises 10 mM histidine, 1.5 M NaCl, pH 7. In another example, the elution solution comprises 5-50 mM HEPES, 1 M to 2 M NaCl, pH 6.8-8.2. In another example, the elution solution comprises 10 mM HEPES, 1.2 M NaCl, pH 7.5. In another example, the elution solution comprises 10 mM HEPES, 1.5 M NaCl, pH 7.5.

In one example, the method comprises diluting the eluted virus at a dilution of 1:5 to 1:20. For example, the method comprises diluting the eluted virus at a dilution of 1:10.

In one example, the method comprises diluting the eluted virus with histidine, Tris, or HEPES. For example, the method comprises diluting the eluted virus with HEPES. In one example, the method comprises diluting the eluted virus with 10 mM HEPES, pH7.5. For example, the virus is diluted immediately after elution.

In one example, the method further comprises incubating the eluted virus for up to 15 minutes at room temperature or up to 60 minutes at 2-8° C.

In one example, the method comprises concentrating and/or diafiltering the eluted virus.

In one example, the method increases the virus infectious titer yield by at least 2% or 3% or 4% or 5% or 10% or 15% or 20%. In one example, the method increases the virus infectious titer yield by at least 10%.

The present disclosure additionally provides a method of purifying an enveloped virus from a cell culture fluid, comprising:
(i) (optionally) providing a cell culture fluid comprising viral vector produced from a stable producer cell line;
(ii) contacting the cell culture fluid with a recombinantly expressed Serratia endonuclease;
(iii) contacting the endonuclease treated cell culture fluid to a filter to produce a filtered cell culture fluid;
(iv) loading the filtered cell culture fluid and a high concentration salt solution comprising 5M sodium chloride on to an anion exchange chromatography membrane, wherein the fluid and the salt solution are loaded at a ratio of 94% (v/v) filtered cell culture fluid and 6% (v/v) high concentration salt solution;
(v) washing the membrane with one or more wash buffers;
(vi) eluting the bound virus from the membrane with an elution buffer comprising 1.2M or 1.5M sodium chloride;
(vii) (optionally) diluting the eluted virus with a buffer; and
(viii) concentrating and diafiltering the eluted virus or the diluted eluted virus.

In one example, the method is performed in-line or continuously or semi-continuously.

In one example, a method of the disclosure additionally comprises performing sterile filtration. For example, the sterile filtration is performed prior to concentrating and diafiltering the eluted virus. In an alternative example, the sterile filtration is performed after concentrating and diafiltering the eluted virus.

In one example, a method of the disclosure additionally provides formulating the enveloped virus into a pharmaceutical formulation or into a solution suitable for infecting a cell.

The disclosure additionally provides a purified enveloped virus produced by a method described herein.

The present disclosure additionally provides a method for preventing fouling of an anion exchanger. Such a method comprises the same steps as set out herein.

In one example, a chromatography method of the disclosure is performed with a substrate having immobilized thereon a positively charged ligand. As will be apparent to the skilled artisan, such a substrate may be an anion exchange chromatography column. Such a substrate may also be a multi-modal chromatography column comprising a positively charged ligand. Thus, the present disclosure additionally encompasses performing a method described herein wherein a multi-modal chromatography column comprising a positively charged ligand is used in place of an anion exchange chromatography column.

### BRIEF DESCRIPTION OF THE DRAWINGS

FIG. 1 is a graphical representation showing that harvest stability is compromised by high salt conditions.

FIG. 2 is a graphical representation showing RNA yield, infectivity yield and filtration capacity of a sterile filter of TFF eluant with or without Benzonase treatment (as indicated).

FIG. 3 is a graphical representation showing the pressure in a Mustang Q anion chromatography column when processing harvested cell culture fluid (A) without or (B) with Benzonase treatment (as indicated).

FIG. 4 is a graphical representation summarizing the yield for infectious titer and RNA content, for harvests collected from flatware and adherent bioreactors.

FIG. 5 is a graphical representation showing recovery of virus from Mustang Q anion exchange performed with or without an in-line 5M NaCl salt spike (as indicated).

FIG. 6 is a graphical representation showing infectious titer yield yield during purification of large scale harvests from adherent bioreactors.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, molecular biology, microbiology, virology).

Unless otherwise indicated, the conventional techniques of molecular biology, microbiology, virology, recombinant DNA technology, peptide synthesis in solution, solid phase peptide synthesis, and immunology utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The term “about”, unless stated to the contrary, refers to +/−20%, more for example +/−10%, of the designated value. For the avoidance of doubt, the term “about” followed by a designated value is to be interpreted as also encompassing the exact designated value itself (for example, “about 10” also encompasses 10 exactly).

As used herein the term “from” in the shall be taken to indicate that a specified integer may be obtained from a particular source albeit not necessarily directly from that source (i.e., includes recombinantly obtained).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

The term “purify” or “purifying” or “purification” shall be taken to mean the removal, whether completely or partially, of at least one impurity present in the cell culture fluid, which thereby improves the level of purity of enveloped virus in solution.

The term “impurity” or “impurities” shall be taken to include one or more components in the cell culture fluid other than the enveloped virus. For example, impurities may include process related impurities such as host cell DNA, host cell proteins, and media components (e.g., fetal bovine serum).

As used herein, the term “enveloped virus” refers to DNA and RNA viruses that have a viral envelope. Envelopes are typically derived from host cell membranes (e.g., phospholipids and proteins), but may include viral glycoproteins on the surface of the envelope. Enveloped viruses also comprise a “capsid”, which is a protein layer between the envelope and viral genome. In one example, the enveloped virus is a retrovirus. For example, the enveloped virus is a lentivirus, e.g., human immunodeficiency virus.

As used herein, the term “cell culture fluid” will be understood to encompass the fluid in which cells are grown for the purpose of producing an enveloped virus. The fluid may comprise the cells or the cells may have been removed, e.g., by centrifugation and/or removal of supernatant.

As used herein, “harvesting” refers to removal of the cell culture media containing virus particles from the producer cells for downstream processing, and “harvest” refers to the cell culture media containing virus particles that has been removed for the purpose of downstream processing. A harvesting process may include collecting one or more harvests. “Harvest filtration” refers to either a harvest that has been filtered or cell culture media containing virus particles that has been filtered to remove the producer cells for downstream processing.

As used herein, the term “filtered cell culture fluid” will be understood to encompass the cell culture fluid after it has been subjected to harvest filtration.

As used herein, the term “salt-spiked cell culture fluid” will be understood to encompass a cell culture fluid or a filtered cell culture fluid that has been mixed with a high concentration salt solution.

The skilled artisan will understand that an “endonuclease” is an enzyme that cleaves the phosphodiester bond within a polynucleotide chain. Endonucleases can cleave DNA or RNA or both DNA and RNA. Endonucleases can cleave in a sequence non-specific manner (also referred to as a “non-specific endonuclease”) or can cleave at specific nucleotide sequences (also referred to as “restriction endonucleases”).

Reference herein to an endonuclease “from” a source, e.g., from Serratia marcescens encompasses the endonuclease purified from that source or produced by other means, e.g., by recombinant techniques.

The term “anion exchange chromatography” specifically includes, without limitation, chromatography performed on anion exchange resins, matrices, absorbers, filters, and the like. For example, the anion exchange chromatography is performed using a positively charged membrane. The skilled artisan will understand that the terms “anion exchange chromatography” and “anion exchange purification” are used interchangeably herein and each term provides explicit support for the other term.

An “anion exchange chromatography column” is a device for separating compounds by anion exchange chromatography. A “column” is a container or vessel used for anion exchange chromatography that contains resins, matrices, adsorbers, filters, and the like, with charged molecules attached. The skilled artisan will understand that the terms “anion exchange chromatography column”, “anion exchange column”, and “anion exchanger” are used interchangeably herein and each term provides explicit support for each other term.

As used herein, the term “immediately after” in the context of performing anion exchange chromatography after harvest filtration means that there are no intervening purification steps between the harvest filtration and the anion exchange. However, this term does not exclude additional steps such as adjusting the pH or adding a salt to the cell culture fluid or filtered cell culture fluid between the harvest filtration and the anion exchange chromatography.

As used herein, the term “high concentration salt solution” will be understood to mean a concentration of salt in excess of 500 mM, such as in excess of 1M, e.g., between 1M and 10M.

As used herein, the term “in-line” in the context of a process step refers to a process step that is integrated into or combined with one or more other process steps, or that flows directly from or to another process step without requiring manual intervention or handling.

As used herein, “in-line mixing”, or “in-line” when used in the context of a mixing step specifically, refers to flowing a first fluid stream comprising a first fluid into contact with a second fluid stream comprising a second fluid, such that the first fluid and the second fluid contact each other and become mixed together. In-line mixing in the context of loading an anion exchange chromatography column includes the two fluid streams contacting each other before entering the anion exchange chromatography column, after entering the anion exchange chromatography column, or simultaneously with entering the anion exchange chromatography column.

### Production of Enveloped Viruses

Methods for the production of enveloped viruses will be apparent to the skilled artisan and/or described, for example, in Ansorge et al., (2010) Biochem. Eng. J. 48:362-377; Schweizer and Merten (2010) Curr. Gene Ther. 10:474-486; and Rodrigues et al., (2011) Viral Gene Therapy. Xu, InTech. Chapter 2:15-40.

In one example, the virus is a retrovirus, for example, a lentivirus. Exemplary retroviruses are from alpha retroviruses (such avian leukosis virus (ALV)), from beta retroviruses (such as mouse mammary tumor virus (MMTV)), from gamma retroviruses (such as murine leukemia virus (MLV)), from delta retroviruses (such as human T-lymphotropic virus (HTLV)), from epsilon retroviruses (such as Walleye dermal sarcoma virus (WDSV)), from spumavirus (such as human foamy virus (HFV) or simian foamy virus (SFV)), from primate lentiviruses such as the different types of human immunodeficiency viruses (HIV), the different types of simian immunodeficiency viruses (SIV), or from non-primate mammal lentiviruses such as the equine infectious anemia virus (EIAV), from the feline immunodeficiency virus (FIV), the caprine arthritis-encephalitis virus (CAEV), or the ovine visna-maedi virus (VMV).

In some examples, the enveloped virus, e.g., the retrovirus, is pseudotyped, i.e., it comprises an envelope glycoprotein derived from a virus different from the virus from which it is derived, a modified envelope glycoprotein or a chimeric envelope glycoprotein.

In some examples, the enveloped virus comprises a transgene introduced into its genome. The transgene will depend on the specific use for which the enveloped viral vector is intended. Exemplary transgenes include a transgene coding for a therapeutic RNA (e.g. encoding an antisense complementary RNA of a target RNA or DNA sequence), a transgene encoding for a protein that is deficient or absent in a subject affected with a pathology, or a transgene used for vaccination with DNA, i.e. a transgene coding for a protein, the expression of which will induce vaccination of the recipient body against said protein. In some examples, the transgene encodes a protein or nucleic acid useful for treating a hemoglobinopathy, e.g., sickle cell disease or a thalassemia. In some examples, the transgene encodes a protein or nucleic acid useful for treating a primary immunodeficiency. In some examples, the transgene encodes a protein or nucleic acid useful for treating Wiskott-Aldrich Syndrome. In some examples, the transgene encodes a protein or nucleic acid useful for treating K linked agammaglobulinemia.

In some examples, an enveloped virus is produced by introducing the four following elements into a host cell: an expression cassette comprising a lentiviral gene gagpol, an expression cassette comprising a lentiviral gene rev, a transgene, all positioned between a lentiviral LTR-5′ and a lentiviral LTR-3′, and an expression cassette encoding envelope glycoprotein(s).

In some examples, the enveloped virus is produced from a stable line expressing one or several elements required for producing an enveloped virus (Miller (2001) Curr. Protoc. Hum. Genet. Chapter 12: Unit 12.5.; Rodrigues et al. 2011, supra). In one example, the enveloped virus is produced from a mammal host cell transfected transiently with one or several plasmids coding for the elements required for producing the virus. According to an alternative example, the elements are introduced into the cell by means of multiple plasmids: one plasmid bearing an expression cassette comprising a lentiviral gagpol gene, one plasmid bearing an expression cassette comprising a lentiviral rev gene, one plasmid bearing an expression cassette encoding the envelope glycoprotein(s), one plasmid bearing an expression cassette comprising a tetracycline transactivator (tTA) gene, and/or one plasmid bearing an expression cassette comprising a lentiviral tat gene. A transfer plasmid comprising an expression cassette with the transgene, comprised between a lentiviral LTR-5′ and LTR-3′, can be introduced as a concatemer along with a helper plasmid with an antibiotic resistance cassette to confer resistance to the producer cells.

The host cell may be selected from any cell allowing production of an enveloped virus. According to one example, the cell is selected from a human cell (HEK293, HEK293T, HEK293FT, HEK293OX, Te671, HT1080, CEM), a musteli cell (NIH-3T3), a mustelidae cell (Mpf), a canid cell (D17). According to one example, the cell is selected from CHO cells, BHK cells, MDCK cells, C3H 10T1/2 cells, FLY I, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, B-50 cells, 3T3 cells, NIH3T3 cells, HepG2 cells, Saos-2 cells, Huh7 cells, HeLa cells, W163 cells, 211 cells, and 211 A cells.

According to one example, the cell is selected from the GPR, GPRG, GPRT, GPRGT, and GPRTG cell lines. In another example, the cell is selected from a cell line derived from any of the above cell lines.

In one example, the enveloped virus is produced from stable producer cells. Stable producer cells can be derived from packaging cell lines, including as any of the cell lines disclosed herein. In some embodiments the packaging cell lines are GPRG or GPRTG cell lines (Throm et al. (2009) Blood 113 (21): 5104-5110; and Bonner et al. (2015) Molecular Therapy, Vol. 23, Suppl. 1, S35). In one example, stable producer cell line cells are generated by synthesizing a vector by cloning one or more genes into a recombinant plasmid; forming a concatemeric array from an expression cassette excised from the synthesized vector, and an expression cassette obtained from an antibiotic resistance cassette plasmid; transfecting packaging cell line cells with the formed concatemeric array; and isolating the stable producer cell line cells. Virus is produced by inducing the inducible promoters of the stable producer cell line cells.

The cells are cultivated in a medium suitable for cultivation of mammal cells and for producing an enveloped virus. The cells can be cultivated in an adherent environment, e.g., while attached to a surface, or in a suspension environment, e.g., suspended in the medium. The medium may moreover be supplemented with additives known in the field such as antibiotics, serum (notably fetal calf serum, etc.) added in suitable concentrations. The medium may be supplemented with GlutaMax™, Pluronic™ F-68 (ThermoFisher), LONG® R3 IGF-I (Sigma-Aldrich), Cell Boost™ 5, and/or an anticlumping agent. The medium used may notably comprise serum or be serum-free. Culture media for mammal cells are known and include, for example, DMEM (Dulbecco's Modified Eagle's medium) medium, RPMI1640 or a mixture of various culture media, including for example DMEM/F12, or a serum-free medium like optiMEM®, optiPRO®, optiPRO-SFM®, CD293® (ThermoFisher), TransFx™ (Cytiva), BalanCD® (Irvine), Freestyle F17® (Life Technologies), or Ex-Cell® 293 (Sigma-Aldrich).

In a process using transiently transfected cells, any agent allowing transfection of plasmids may be used. Exemplary agents include calcium phosphate or polyethyleneimine. The conditions (e.g., amount of plasmid(s), ratio between the plasmids, ratio between the plasmid(s) and the transfection agent, the type of medium, etc.) and the transfection time may be adapted by one skilled in the art according to the characteristics of the produced virus and/or of the transgene introduced into the transfer plasmid.

According to some examples, the culture medium used has a neutral pH (e.g. comprised between 7 and 7.4, notably 7, 7.1, 7.2, 7.3 or 7.4) conventionally used in the state of the art for cultivating cells and producing viruses. In other examples, the production process used comprises the cultivation of producing cells in a moderately acid medium. The expression “moderately acid condition” designates the pH of an aqueous solution comprised between 5 and 6.8, for example between 5.5 and 6.5, such as between 5.8 and 6.2. The selected pH will also depend on the buffering power of the culture medium used, which one skilled in the art may easily determine taking into account his/her general knowledge. One skilled in the art is able to modify the pH of a solution.

In one example, the production of the enveloped virus comprises: transient transfection of HEK293T cells or derivatives thereof by means of one or several plasmids coding for the elements required for production of said enveloped vector, or by the use of stable producing cells, e.g., GPRG or GPRTG, producing the vectors constitutively or after induction; culturing the cells in a suitable medium, for which the pH is of about 6 or of about 7; harvesting cell culture medium containing the enveloped virus.

### Purifying Enveloped Viruses

The present disclosure provides methods for improving the purity and/or recovery of enveloped viruses from cell culture fluid or filtered cell culture fluid.

Methods of the disclosure are applicable to purifying enveloped viruses from both small- and large-scale productions. The methods are particularly useful for their ability to be scaled up for manufacturing pharmaceutical products at commercial scale. In one example, cells are grown in an adherent or fixed-bed environment. In one example, cells are grown in a cell culture chamber, such as a CellSTACK® (Corning). In one example, cells are grown in an adherent bioreactor, such as iCELLis® (Pall), scale-XTM or NevoLine™ (Univercells Technologies). An adherent cell culture chamber or bioreactor may have an available growth surface of greater than about 0.1 m2, greater than about 1 m², greater than about 10 m², greater than about 30 m2, greater than about 100 m², greater than about 200 m², greater than about 500 m², or greater than about 600 m².

In one example, cells are grown in a suspension environment. In one example, cells are grown in a stirred tank bioreactor. In examples, the cells are grown in a Biostat® or Univessel® bioreactor (Sartorius).

In embodiments, the volume of a harvest of cell culture fluid can be for example, about 0.01 L to about 0.1 L, or about 0.1 L to about 1 L, or about 1 L to about 5 L. For example, the volume of a harvest of cell culture fluid is about 5 L. In other embodiments, the volume of a harvest of cell culture fluid can be about 5 L to about 10 L, about 10 L to about 50 L, about 50 L to about 100 L, about 100 L to about 200 L, about 200 L to about 500 L, about 500 L to about 1000 L, about 1000 L to about 2000 L, or about 2000 L to about 5000 L. In one example, the volume of the harvest is between about 35 and 150 L. In one example, the volume of the harvest is about 35-150 L. For example, the volume of the harvest is about 20 L. In one example, the volume of the harvest is about 50-70 L. For example, the volume of the harvest is about 50 L.

The downstream process for purifying and concentrating viral vector from a cell culture fluid includes a harvest filtration step (also known as “clarification filtration” or “harvest clarification filtration” or “bioburden reduction”) to remove cellular debris and components from the harvest, a purification step, e.g., anion exchange chromatography, to reduce overall volume and to separate viral vector from host cell DNA, proteins, and media components, and an ultrafiltration/diafiltration step to concentrate the viral vector into a final formulation buffer. In some examples, the downstream step further includes a sterile filtration step for removal of microorganisms from the final product.

### Endonuclease Treatment

An endonuclease treatment of the cell culture fluid or filtered cell culture fluid was added to the downstream process. The endonuclease treatment can occur during or after harvesting the cell culture fluid, but before anion exchange chromatography. For example, endonuclease can be added directly to a bag or other vessel into which the harvested cell culture fluid is collected. In another example, endonuclease is added to the cell culture fluid after collection but before harvest filtration. In another example, endonuclease is added to the cell culture fluid after harvest filtration but before anion exchange chromatography. In another example, endonuclease is mixed in-line with the cell culture fluid when loading the anion exchange chromatography column, such that the endonuclease contacts the cell culture fluid immediately prior to entering the column or after entering the column.

In embodiments in which the endonuclease is contacted to the cell culture fluid prior to loading onto the anion exchange chromatography column, the endonuclease can be incubated with the cell culture fluid for up to about 30 hours. For example, the cell culture fluid with endonuclease can be stored for about 22 hours, about 6 hours, about 4 hours about 2 hours, or about 1 hour.

In embodiments in which the endonuclease is contacted to the cell culture fluid during harvesting or prior to the harvest filtration step, optionally no additional incubation step is required, as the incubation effectively occurs during the intervening process steps prior to loading of the cell culture fluid onto the anion exchange chromatography column.

In one example, a harvested cell culture fluid is filtered following production of the enveloped virus. Prior to harvest filtration, the cell culture fluid is contacted with an endonuclease. The inventors identified that contacting the cell culture fluid with endonuclease prior to harvest filtration reduces clogging of the filters and subsequent purification processes, e.g., anion exchange chromatography. The inventors additionally found that contacting the cell culture fluid with an endonuclease permitted sterile filtration of a purified enveloped virus without clogging the filter. The inventors additionally found that treating the cell culture fluid with an endonuclease did not significantly reduce the infectivity of the purified enveloped virus or total RNA yield.

In one example, the cell culture fluid is contacted with the endonuclease prior to harvest filtration, i.e., filtration to remove cells and cellular debris. In one example, the harvest filtration is performed using membrane filtration. For example, the harvest filtration is performed using a 0.8 μm filter and a 0.45 μm filter, which may be included within a single unit.

In one example, the cell culture fluid is contacted with the endonuclease for about 1-4 hours. For example, the cell culture fluid is contacted with the endonuclease for about 1-3 hours. In one example, the cell culture fluid is contacted with the endonuclease for about 1 hour. In some examples, the endonuclease is added to the cell culture medium and the harvest filtration commenced without any additional incubation time.

Suitable endonucleases will be apparent to the skilled artisan based on the disclosure herein. In one example, the endonuclease cleaves in a sequence non-specific manner. For example, the endonuclease cleaves DNA (and, optionally RNA) into short oligonucleotides, e.g., 2-10 bp long, such as 3-7 bp long, e.g., 3-5 bp long.

In one example, the endonuclease is from Serratia marcescens, Anabaena sp., Saccharomyces cerevisiae, Bos Taurus, Syncephalostrum racemosum and/or Borrelia burgdorferi.

In one example, the endonuclease is a Serratia nuclease, NucA, Nucl and/or endonuclease G.

The endonuclease may be isolated or purified from the recited source. Alternatively, the endonuclease can be produced recombinantly.

The endonuclease can also be obtained from a suitable commercial source, as will be apparent to the skilled artisan and/or described herein. For example, endonucleases are available from New England Biolabs, Inc or c-LEcta GmbH.

In one example, the endonuclease is from Serratia marcescens. Such an endonuclease is also referred to as Golden nuclease. This nuclease is sold under the tradenames Benzonase® or Denarase®.

In one example, the method involves adjusting the concentration of Mg²⁺ in the cell culture fluid to achieve a concentration of up to 10 mM Mg²⁺. In one example, the concentration of Mg²⁺ is adjusted to about 1-2 mM. In one example, the concentration of Mg²⁺ is adjusted to 2 mM. In one example, the concentration of Mg²⁺ in the cell culture fluid is about 0.8 mM and is not adjusted further.

In one example, the method involves adjusting the pH of the cell culture fluid to 6.0 to 10.0. In one example, the method involves adjusting the pH of the cell culture fluid to 8.0 to 9.2.

In one example, the cell culture fluid is at a temperature of between 0° C. and 42° C. during contact with the endonuclease. In one example, the cell culture fluid is at a temperature of between 2° C. and 8° C. during contact with the endonuclease. In one example, the cell culture fluid is at a temperature of 4° C. during contact with the endonuclease. In one example, the cell culture fluid is at a temperature of between 35° C. and 40° C. during contact with the endonuclease. In one example, the cell culture fluid is at a temperature of about 37° C. during contact with the endonuclease. In one example, the cell culture fluid is at a temperature of between 18° C. and 22° C. during contact with the endonuclease. In one example, the cell culture fluid is at a temperature of 20° C. during contact with the endonuclease.

In one example, the concentration of dithiothreitol (DTT) in the cell culture fluid is 0-100 mM. In one example, the concentration of 2-Mercaptoethanol in the cell culture fluid is 0-100 mM. In one example, the concentration of monovalent cations, e.g., Na⁺ or K⁺ in the cell culture fluid is 0-150 mM. In one example, the concentration of monovalent cations, e.g., Na⁺ or K⁺ in the cell culture fluid is 0-20 mM. In one example, the concentration of PO₄³⁻ in the cell culture fluid is 0-100 mM. In one example, the concentration of PO₄³⁻ in the cell culture fluid is 0-10 mM.

In one example, the endonuclease is added to the culture medium at a concentration of 0.001U/mL cell culture medium to 100U/mL cell culture medium. For example, the endonuclease is added to the cell culture medium at a concentration of 0.01U/mL cell culture medium to 10U/mL cell culture medium. For example, the endonuclease is added to the cell culture medium at a concentration of 0.1U/mL cell culture medium to 1U/mL cell culture medium. For example, the endonuclease is added to the cell culture medium at a concentration of less than 0.5U/mL cell culture medium.

In one example, the endonuclease is added to the cell culture medium at a concentration of 0.3U/mL cell culture medium.

In some examples, the pH of the cell culture fluid is not adjusted prior to treatment with the endonuclease.

In some examples, the endonuclease is diluted prior to addition to the cell culture medium. For example, the endonuclease is diluted in the medium in which the cells are grown. For example, the endonuclease is diluted in DMEM. In one example, the endonuclease is diluted in a buffer or medium in the absence of fetal bovine serum (FBS). For example, the endonuclease is diluted in a buffer comprising HEPES.

Following treatment with the endonuclease, the cell culture fluid is filtered.

### Anion Exchange Purification

Following harvest filtration, the enveloped virus is purified using anion exchange.

In one example, the anion exchange is performed in bind-elute mode. In this regard, the enveloped virus binds to the anion exchanger while contaminants flow through. The virus is subsequently eluted from the anion exchanger. Performing anion exchange in this manner reduces the volume of liquid in which the virus is suspended and removes contaminants such as host cell DNA, host cell proteins, and medium components like fetal bovine serum.

Suitable anion exchangers will be apparent to the skilled artisan. Exemplary anion exchangers are a column comprising a resin or a membrane or another suitable substrate.

In one example, the anion exchanger is a membrane anion exchanger.

In one example, the anion exchanger is a weak anion exchanger, e.g., comprising an ion exchange group selected from a diethylaminoethyl (DEAE) or aminoethyl group.

In another example, the anion exchanger is a strong anion exchanger, e.g., comprising an ion exchange group selected from a quaternary ammonium (Q), diethyl-2-hydroxypropylaminoethyl (QAE), triethylaminoethyl (TEAE), or trimethyl aminoethyl group. Exemplary anion exchangers useful in the method of the present disclosure include MUSTANG® E, MUSTANG® Q, SARTOBIND® Q, CHROMASORB®, POSSIDYNE®, CAPTOR Q, QSFF, POROS® Q, FRACTOGEL® Q, NATRIX® Q.

As exemplified herein, the anion exchanger comprises a Q ion exchange group.

As exemplified herein, the anion exchanger is a membrane anion exchanger comprising a Q ion exchange group. For example, the anion exchanger is MUSTANG® Q.

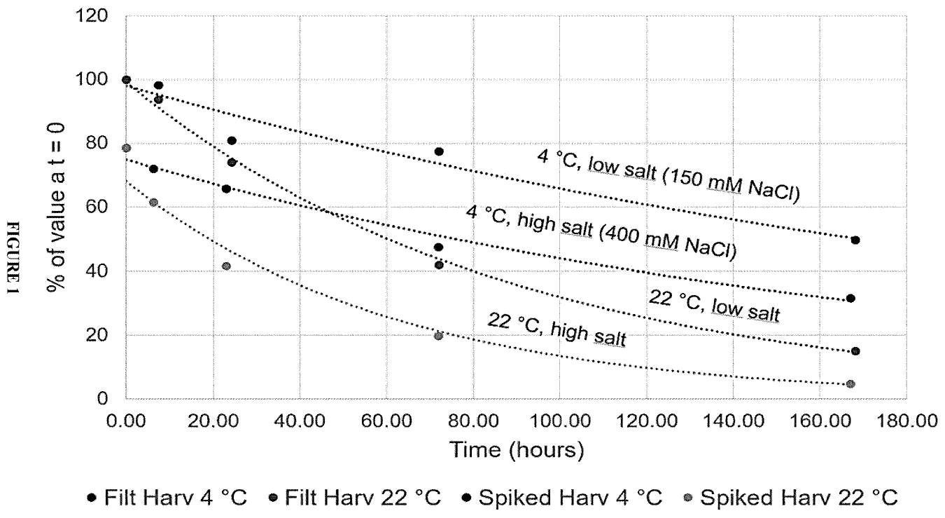

As discussed herein, the inventors determined that the salt concentration in cell culture medium or filtered cell culture medium is too low for effective anion exchange chromatography. For example, the salt concentration of the harvested cell culture medium identified by the inventors was about 150 mM. The inventors determined that a final salt concentration in the cell culture medium or filtered cell culture medium of 300-500 mM, e.g., about 400 mM was desirable for loading onto the anion exchange chromatography column. A salt concentration of about 400 mM reduced impurities binding to the anion exchanger. To increase the salt concentration required adding (or “spiking”) the cell culture medium or filtered cell culture medium with a salt solution to generate a salt-spiked cell culture medium. The inventors wished to avoid adding too much volume to the cell culture medium or filtered cell culture medium, and therefore it was desirable to use a high-concentration salt solution. However they also recognized that addition of salt to the cell culture medium or filtered cell culture medium causes the virus to become unstable. FIG. 1 shows that harvest stability decreases under high salt conditions, as compared to low salt conditions. Immediately after addition of salt, titer decrease by about 20-30%. Without wishing to be bound by theory, the inventors suggest that this initial loss of titer may be caused by formation of local areas of high salt when mixing with a high-concentration salt solution.

This problem is compounded by the difficulty of mixing the cell culture medium or filtered cell culture medium with the high-concentration salt solution quickly enough to avoid prolonged contact of the virus with high salt concentrations without using more vigorous mixing techniques that would destabilize the virus by exposure to high shear forces. Uneven or insufficient mixing not only harms the virus, but it also hampers the anion exchange process itself by causing virus to elute during loading.

Typical methods of mixing involve manual handling steps and are time-consuming at commercial scale. To achieve adequate mixing using traditional methods at commercial scale could add several hours of process time.

The inventors' solution to these problems is to contact the cell culture medium or filtered cell culture medium with a high concentration salt solution in an in-line process during loading onto the anion exchange chromatography column. This method achieved improved mixing, without manual handling steps or adding any process time, and can be performed in-line in a continuous or semi-continuous manner, thereby streamlining the downstream process.

For example, the the high concentration salt solution and the filtered cell culture fluid or the cell culture fluid are mixed to generate a salt-spiked cell culture medium during loading on to the anion exchange chromatography column.

For example, the high concentration salt solution is added to the anion exchange chromatography column while the cell culture medium or filtered cell culture medium is being loaded onto the column.

In one example, contacting the cell culture medium or filtered cell culture medium with a high concentration salt solution during loading involves flowing two fluid streams (one containing the cell culture medium or filtered cell culture medium and the other containing the high concentration salt solution) together into one fluid stream. Flowing the fluid streams together causes them to mix to generate a salt-spiked cell culture medium, either as they are being loaded onto an anion exchanger, immediately before loading onto the anion exchanger, or within the anion exchanger. Whether performed at lab scale or commercial scale, the method does not add substantial process time because it is performed in-line during loading of the anion exchanger.

In one example, the salt in the high concentration salt solution is monovalent or divalent. For example, the salt in the high concentration salt solution is monovalent. In one example, the salt in the high concentration salt solution is NaCl or KCl. In an exemplified form of the disclosure, the salt in the high concentration salt solution is NaCl.

In one example, the concentration of salt in the high concentration salt solution is between 1M and 10M. For example, the concentration of salt in the high concentration salt solution is between 2M and 8M. For example, the concentration in the high concentration salt solution is between 3M and 7M. For example, the concentration of salt in the high concentration salt solution is 5M.

In one example, the high concentration salt solution is 5M NaCl.

In one example, the high concentration salt solution is added to achieve a final concentration of the salt of 300 mM to 500 mM in the salt-spiked cell culture medium for loading onto the anion exchange chromatography column. For example, the high concentration salt solution is added to achieve a salt-spiked cell culture medium with a final concentration of the salt of 400 mM.

In one example, following loading, the anion exchange chromatography column is washed with a wash solution comprising a buffer and a salt. For example, the buffer is histidine, HEPES, or Tris. For example, the salt is a monovalent salt, e.g., NaCl.

In one example, the wash solution comprises 5-50 mM histidine, 150 mM NaCl, pH 5.5-7.4, for example 10 mM histidine buffer, 150 mM NaCl, pH 7. In another example the wash solution comprises 10-100 mM Tris, 150 mM NaCl, pH 7.0-9.0, for example 50 mM Tris, 150 mM NaCl, pH 8. In another example, the wash solution comprises 5-50 mM HEPES, 150 mM NaCl, pH 6.8-8.2, for example 10 mM HEPES, 150 mM NaCl, pH 7.5.

In one example, the wash solution comprises 5-50 mM histidine, 750 mM NaCl, pH 5.5-7.4, for example 10 mM histidine buffer, 750 mM NaCl, pH 7. In another example the wash solution comprises 10-100 mM Tris, 750 mM NaCl, pH 7.0-9.0, for example 50 mM Tris, 750 mM NaCl, pH 8. In another example, the wash solution comprises 5-50 mM HEPES, 750 mM NaCl, pH 6.8-8.2, for example 10 mM HEPES, 750 mM NaCl, pH 7.5.

In one example, the conductivity of the wash solution is 10 mS/cm-20 mS/cm.

In one example, following loading, the anion exchange chromatography column is washed with a first wash solution comprising a buffer and a salt and a second wash solution comprising a buffer and a salt. For example, the buffer is histidine, HEPES, or Tris. For example, the salt is a monovalent salt, e.g., NaCl.

In one example, the first and second wash solutions comprise the same buffer and same salt, however the second wash solution comprises a higher concentration of salt than the first wash solution.

In one example, the conductivity of the second wash solution is 60 mS/cm-75 mS/cm.

In one example, the first wash solution comprises 5-50 mM histidine, 150 mM NaCl, pH 5.5-7.4, for example 10 mM histidine buffer and 150 mM NaCl, pH 7; and the second wash solution comprises 5-50 mM histidine, 750 mM NaCl, pH 6.0-8.0, for example 10 mM histidine buffer and 750 mM NaCl, pH 7.

In one example, the first wash solution comprises 10-100 mM Tris, 150 mM NaCl, pH 7.0-9.0, for example 50 mM Tris and 150 mM NaCl, pH 8; and the second wash solution comprises 10-100 mM Tris, 750 mM NaCl, pH 7.0-9.0, for example 50 mM Tris and 750 mM NaCl, pH 8.

In one example, the first wash solution comprises 5-50 mM HEPES, 150 mM NaCl, pH 6.8-8.2, for example 10 mM HEPES, 150 mM NaCl, pH 7.5; and the second wash solution comprises 5-50 mM HEPES, 750 mM NaCl, pH 6.8-8.2, for example 10 mM HEPES, 750 mM NaCl, pH 7.5.

Following washing, the method can comprise eluting the enveloped virus. In one example, the virus is eluted with a solution comprising a buffer and a salt. For example, the buffer is histidine, HEPES, or Tris. For example, the salt is a monovalent salt, e.g., NaCl.

In one example, the first and second wash and elution solutions comprise the same buffer and same salt, however the elution solution comprises a higher concentration of salt than the first and second (if used) wash solution.

In one example, the elution solution comprises 5-50 mM histidine, 1 M to 2 M NaCl, pH 5.5-7.4, for example 10 mM histidine buffer and 1200 or 1500 mM NaCl. In another example, the elution solution comprises 10-100 mM Tris, 1 M to 2 M NaCl, pH 7.0-9.0, for example 50 mM Tris and 1200 or 1500 mM NaCl, pH 8. In another example, the elution solution comprises 5-50 mM HEPES, 1 M to 2 M NaCl, pH 6.8-8.2, for example 10 mM HEPES, 1200 or 1500 mM NaCl, pH 7.5.

In one example, the conductivity of the elution solution is 110-130 mS/cm.

In one example, the pH of a histidine containing solution is 7.

In one example, the pH of a Tris containing solution is 8.

In one example, the pH of a HEPES containing solution is 7.5.

In one example, the anion exchange chromatography comprises:
(i) loading with cell culture fluid or filtered cell culture fluid and 5M NaCl;
(ii) washing with 10 mM histidine and 150 mM NaCl;
(iii) washing with 10 mM histidine and 750 mM NaCl; and
(iv) eluting with 10 mM histidine and 1500 mM NaCl.

In another example, the anion exchange chromatography comprises:
(i) loading with cell culture fluid or filtered cell culture fluid and 5M NaCl;
(ii) washing with 50 mM Tris and 150 mM NaCl;
(iii) washing with 50 mM Tris and 750 mM NaCl; and
(iv) eluting with 50 mM Tris and 1500 mM NaCl.
In another example, the anion exchange chromatography comprises:
(i) loading with cell culture fluid or filtered cell culture fluid and 5M NaCl;
(ii) washing with 10 mM HEPES and 150 mM NaCl;
(iii) washing with 10 mM HEPES and 750 mM NaCl; and
(iv) eluting with 10 mM HEPES and 1500 mM NaCl.

In one example, following elution, the resulting eluate is diluted to reduce the salt concentration, either by mixing the eluate with a dilution buffer in-line, or by eluting directly into the dilution buffer, or by eluting and diluting in separate steps. For example, the eluate is diluted with a solution comprising or consisting of histidine buffer (e.g., comprising 10 mM L-histidine) or Tris (e.g., comprising 50 mM Tris) or HEPES (e.g., comprising 10 mM HEPES). For example, the eluate is diluted with a solution comprising the same buffer used to elute the virus. For example, the eluate is diluted 1:10 with the solution if elution was done with 1500 mM NaCl or the eluate is diluted 1:8 with the solution if elution was done with 1200 mM NaCl. In one example, the eluate is diluted 1:10 with a solution comprising 10 mM HEPES, pH 7.5.

Throughout the specification, reference is made to anion exchange chromatography. It should be understood that the methods described herein could be applied to other ion exchange chromatography as well. For example, cation exchange chromatography could be used to bind impurities while viral vector flows through. The person of ordinary skill in the art could readily modify the disclosed methods to suit other ion exchangers as needed.

### Additional Steps

In one example, an enveloped virus eluted from anion exchange column is further purified on the basis of its size. In one example, the buffer in which virus was eluted from the anion exchange column, is exchanged more or less at the same time. In the process of the disclosure, tangential flow filtration is preferred. This method permits impurity removal and buffer exchange at almost the same time.

Tangential flow ultrafiltration/diafiltration is a method which may be used to remove residual protein and nucleic acids as well as for exchanging working buffer into a final formulation buffer. Ultrafiltration using tangential flow is preferred and different devices can be used (e.g. Proflux and LABSCALE (ultrafiltration system) TFF System, both Millipore or the KR2i system from Repligen). The particular ultrafiltration membrane selected will be of a filter pore size sufficient small to retain enveloped virus but large enough to allow penetration of impurities. Depending on the manufacturer and membrane type, nominal molecular weight cut-offs between 100 and 1000 kDa may be appropriate (e.g. UFP-750-E-5A, GE Healthcare; BIOMAX. (ultrafiltration device) NMWC 1000, Millipore). In one example, the molecular weight cut-off is 500 kDa. The membrane composition may be, but it is not limited to, regenerate cellulose, (modified) polyethersulfone, polysulfone. Membranes can be of flat sheet or hollow fibre type. The main parameters that must be optimized are flux rate and trans-membrane pressure. In combination with nominal molecular weight cut-off these two parameters will enable efficient purification and buffer exchange and high virus yield.

As an additional step sterile filtration may be performed to eliminate bioburden. Therefore diluted eluate or final retentate from the ultrafiltration step may be filtered through a filter, for example a 0.22 μm filter. The filter may be constructed from various materials, which may include but are not limited to polypropylene, hydrophilic PVDF, cellulose, hydrophilic regenerated cellulose, cellulose esters, wetting agent-free cellulose acetate, cellulose acetate, nylon, hydrophilic nylon membrane, polyethersulfone, hydrophilic polyethersulfone, hydrophilic asymmetric PES, or any other material which is consistent with low unspecific influenza virus binding. The filter may have a single membrane layer or more than one layer or may incorporate a prefilter of the same or different material, for example a 0.45 μm prefilter. The sterile filtrated virus can be held frozen for subsequent manipulation.

In one example, the sterile filter has a filtration area of at least 15 cm². For example, the sterile filter has a filtration area of about 17.8 cm² or about 20 cm². In one example, the sterile filter has a filtration area of at least 200 cm². For example, the sterile filter has a filtration area of about 210 cm² or about 220 cm².

In one example, the sterile filter has a filtration capacity of at least 2.5 mL/cm². For example, the sterile filter has a filtration capacity of at least 4.0 mL/cm².

The invention is further disclosed in the following numbered paragraphs:
1. A method of purifying an enveloped virus from a cell culture fluid or a filtered cell culture fluid, comprising contacting the cell culture fluid or the filtered cell culture fluid with an endonuclease prior to purifying the virus.
2. The method of paragraph 1, wherein the cell culture fluid is harvested from stable producer cells.
3. The method of paragraphs 1 or 2, wherein the endonuclease is a non-specific endonuclease and degrades both DNA and RNA without sequence specificity.
4. The method of any of paragraphs 1 to 3, wherein the endonuclease is from Serratia marcescens, Anabaena sp., Saccharomyces cerevisiae, Bos Taurus, Syncephalostrum racemosum and/or Borrelia burgdorferi.
5. The method of any one of paragraphs 1 to 4, wherein the endonuclease is a Serratia nuclease, NucA, Nuc1, endonuclease G, DNase I, or micrococcal nuclease.
6. The method of any one of paragraphs 1 to 5, wherein the endonuclease is contacted to the cell culture fluid or the filtered cell culture fluid at a concentration of 0.001 to 100 units/mL of cell culture fluid or filtered cell culture fluid.
7. The method of paragraph 6, wherein the endonuclease is contacted to the cell culture fluid or the filtered cell culture fluid at a concentration of 0.01 to 10 units/mL of cell culture fluid or filtered cell culture fluid.
8. The method of paragraph 7, wherein the endonuclease is contacted to the cell culture fluid or the filtered cell culture fluid at a concentration of 0.1 to 1 unit/mL of cell culture fluid or filtered cell culture fluid.
9. The method of paragraph 8, wherein the endonuclease is contacted to the cell culture fluid or the filtered cell culture fluid at a concentration of about 0.3 units/mL of cell culture fluid or filtered cell culture fluid.
10. The method of any of paragraphs 1 to 9, wherein purification is performed less than about 30 hours after contacting the cell culture fluid or the filtered cell culture fluid with the endonuclease, less than about 22 hours after contacting the cell culture fluid or the filtered cell culture fluid with the endonuclease, less than about 6 hours after contacting the cell culture fluid or the filtered cell culture fluid with the endonuclease, less than about 4 hours after contacting the cell culture fluid or the filtered cell culture fluid with the endonuclease, less than about 2 hours after contacting the cell culture fluid or the filtered cell culture fluid with the endonuclease, less than about 1 hour after contacting the cell culture fluid or the filtered cell culture fluid with the endonuclease, or less than about 30 minutes after contacting the cell culture fluid or the filtered cell culture fluid with the endonuclease.
11. The method of any of paragraphs 1 to 10, wherein the endonuclease is contacted to the cell culture fluid or the filtered cell culture fluid at a concentration of about 0.3 units/mL of cell culture fluid or filtered cell culture fluid, and purification is performed between about 1 and about 2 hours after contact.
12. The method of any of paragraphs 1 to 10, wherein the endonuclease is contacted to the cell culture fluid or the filtered cell culture fluid at a concentration of between about 0.01 and 0.3 units/mL of cell culture fluid or filtered cell culture fluid, and purification is performed at greater than about 2 hours after contact.
13. The method of any of paragraphs 1-10, wherein the endonuclease is contacted to the cell culture fluid or the filtered cell culture fluid at a concentration of between about 0.3 and 10 units/mL of cell culture fluid or filtered cell culture fluid, and purification is performed at less than about 1 hour after contact.
14. The method of any of paragraphs 1 to 10 or 13, wherein the endonuclease is contacted to the cell culture fluid or the filtered cell culture fluid immediately prior to purification.
15. The method of any one of paragraphs 1 to 14, wherein the method comprises performing a harvest filtration on the cell culture fluid to produce the filtered cell culture fluid before contacting the filtered cell culture fluid with the endonuclease.
16. The method of any one of paragraphs 1 to 14, wherein the method comprises performing a harvest filtration after contacting the cell culture fluid with the endonuclease to produce the filtered cell culture fluid.
17. The method of paragraph 16, wherein the harvest filtration is performed immediately after contacting the cell culture fluid with the endonuclease.
18. The method of any one of paragraphs 1 to 17, wherein purification comprises subjecting the filtered cell culture fluid to anion exchange chromatography.
19. The method of paragraph 18, wherein the filtered cell culture fluid is subjected to anion exchange chromatography immediately after harvest filtration.
20. The method of paragraph 19, wherein the filtered cell culture fluid is contacted with a high concentration salt solution to form a salt-spiked cell culture fluid prior to or during loading on to the anion exchange chromatography column.
21. A method of purifying an enveloped virus from a filtered cell culture fluid using anion exchange chromatography, wherein the filtered cell culture fluid is contacted with a high concentration salt solution to form a salt-spiked cell culture fluid prior to or during loading on to the anion exchange chromatography column.
22. The method of paragraph 20 or 21, wherein the filtered cell culture fluid is contacted with a high concentration salt solution to form a salt-spiked cell culture fluid immediately prior to loading on to the anion exchange chromatography column
23. The method of any one of paragraphs 20 or 22, wherein the high concentration salt solution and the filtered cell culture fluid are mixed in-line.
24. The method of any one of paragraphs 20 to 23, wherein the high concentration salt solution comprises a monovalent and/or a divalent salt.
25. The method of paragraph 24, wherein the monovalent salt is sodium chloride.
26. The method of any one of paragraphs 20 to 25, wherein the high concentration salt solution is at a concentration of at least about 1M.
27. The method of any one of paragraphs 20 to 26, wherein the filtered cell culture fluid and the high concentration salt solution are mixed at a ratio of about 70-99% (v/v) filtered cell culture fluid and about 1-30% (v/v) high concentration salt solution.
28. The method of any one of paragraphs 20 to 27, wherein the high concentration salt solution is at a concentration of about 1M.
29. The method of paragraph 28, wherein the filtered cell culture fluid and the high concentration salt solution are mixed at a ratio of about 70% (v/v) filtered cell culture fluid and about 30% (v/v) high concentration salt solution.
30. The method of any one of paragraphs 20 to 27, wherein the high concentration salt solution is at a concentration of 2M.
31. The method of paragraph 30, wherein the filtered cell culture fluid and the high concentration salt solution are mixed at a ratio of about 85% (v/v) filtered cell culture fluid and about 15% (v/v) high concentration salt solution.
32. The method of any one of paragraphs 20 to 27, wherein the high concentration salt solution is at a concentration of 5M.
33. The method of paragraph 32, wherein the filtered cell culture fluid and the high concentration salt solution are mixed at a ratio of about 94% (v/v) filtered cell culture fluid and about 6% (v/v) high concentration salt solution.
34. The method of any one of paragraphs 20 to 27, wherein the high concentration salt solution is at a concentration of 10M.
35. The method of paragraph 34, wherein the filtered cell culture fluid and the high concentration salt solution are mixed at a ratio of about 97% (v/v) filtered cell culture fluid and about 3% (v/v) high concentration salt solution.
36. The method of any one of paragraphs 20 to 35, wherein the salt-spiked cell culture fluid has a salt concentration of between 300 and 500 mM.
37. The method of paragraph 36, wherein the salt-spiked cell culture fluid has a salt concentration of about 400 mM.
38. The method of any one of paragraphs 20 to 37, wherein the salt-spiked cell culture fluid has a target conductivity of 35 to 45 mS/cm at 25° C.
39. The method of any one of paragraphs 20 to 38, wherein the salt-spiked cell culture fluid has a target conductivity of 36 to 44 mS/cm at 25° C.
40. The method of paragraph 38 or 39, wherein the salt-spiked cell culture fluid has a target conductivity of 40 mS/cm at 25° C.
41. The method of any one of paragraphs 18 to 40, wherein the method further comprises washing the anion exchange chromatography column with one or more wash steps.
42. The method of paragraph 41, wherein the method comprises a first wash step with a first wash solution comprising: 10-100 mM Tris, 150 mM NaCl, pH 7.0-9.0; 5-50 mM histidine, 150 mM NaCl, pH 5.5-7.4; or 5-50 mM HEPES, 150 mM NaCl, pH 6.8-8.2.
43. The method of paragraph 42, wherein the first wash solution comprises: 50 mM Tris, 150 mM NaCl, pH 8; 10 mM histidine, 150 mM NaCl, pH 7; or 10 mM HEPES, 150 mM NaCl, pH 7.5.
44. The method of any one of paragraphs 41 to 43, wherein the method comprises a second wash step with a second wash solution comprising: 10-100 mM Tris, 750 mM NaCl, pH 7.0-9.0; 5-50 mM histidine, 750 mM NaCl, pH 5.5-7.4; or 5-50 mM HEPES, 750 mM NaCl, pH 6.8-8.2.
45. The method of paragraph 44, wherein the second wash solution comprises: 50 mM Tris, 750 mM NaCl, pH 8; 10 mM Tris, 750 mM NaCl, pH 7; or 10 mM HEPES, 750 mM NaCl, pH 7.5.
46. The method of any one of paragraphs 18 to 45, wherein the method further comprises eluting bound virus from the anion exchange chromatography column with an elution solution.
47. The method of paragraph 46, wherein the elution solution comprises: 10-100 mM Tris, 1 M to 2 M NaCl, pH 7.0-9.0; 5-50 mM histidine, 1 M to 2 M NaCl, pH 5.5-7.4; or 5-50 mM HEPES, 1 M to 2 M NaCl, pH 6.8-8.2.
48. The method of paragraph 47, wherein the elution solution comprises: 50 mM Tris, 1.2 M or 1.5M NaCl, pH 8; 10 mM histidine, 1.2 M or 1.5M NaCl, pH 7; or 10 mM HEPES, 1.2 or 1.5 M NaCl, pH 7.5.
49. The method of any one of paragraphs 46 to 48, wherein the method further comprises diluting the eluted virus with histidine, Tris, or HEPES.
50. The method of any one of paragraphs 46 to 49, wherein the method further comprises incubating the eluted virus or the diluted eluted virus for up to 15 minutes at room temperature or up to 60 minutes at 2-8° C.
51. The method of any one of paragraphs 46 to 50, wherein the method further comprises concentrating and/or diafiltering the eluted virus or the diluted eluted virus.
52. The method of any one of paragraphs 18 to 51, wherein the anion exchange chromatography column is an anion exchange membrane adsorber.
53. The method of any one of paragraphs 1 to 52, wherein the method increases the virus infectious titer yield by at least 10%.
54. The method of any one of paragraphs 1 to 53, wherein the enveloped virus is a retrovirus.
55. The method of paragraph 54, wherein the retrovirus is a lentivirus.
56. A method of purifying an enveloped virus from a cell culture fluid, comprising:
   (i) providing a cell culture fluid comprising viral vector produced from a stable producer cell line;
   (ii) contacting the cell culture fluid with a recombinantly expressed Serratia endonuclease;
   (iii) contacting the endonuclease treated cell culture fluid to a filter to produce a filtered cell culture fluid;
   (iv) loading the filtered cell culture fluid and a high concentration salt solution comprising 5M sodium chloride on to an anion exchange chromatography membrane, wherein the fluid and the salt solution are loaded at a ratio of 94% (v/v) filtered cell culture fluid and 6% (v/v) high concentration salt solution;
   (v) washing the membrane with one or more wash buffers;
   (vi) eluting the bound virus from the membrane with an elution buffer comprising 1.2M or 1.5M sodium chloride;
   (vii) diluting the eluted virus with a buffer; and
   (viii) concentrating and diafiltering the eluted virus.
57. The method of any one of paragraphs 1 to 56, wherein the cell culture fluid and/or the filtered cell culture fluid has a volume of greater than about 1 L, about 5 L, about 10 L, about 50 L, about 100 L, about 500 L, or about 1000 L.
58. The method of any one of paragraphs 1 to 57, wherein the cell culture fluid and/or the filtered cell culture fluid has a volume of about 5 L.
59. The method of any one of paragraphs 1 to 57, wherein the cell culture fluid and/or the filtered cell culture fluid has a volume of about 20 L.
60. The method of any one of paragraphs 1 to 57, wherein the cell culture fluid and/or the filtered cell culture fluid has a volume of about 44 L.
61. The method of any one of paragraphs 1 to 57, wherein the cell culture fluid and/or the filtered cell culture fluid has a volume of about 60 L.
62. The method of any one of paragraphs 1 to 57, wherein the cell culture fluid and/or the filtered cell culture fluid has a volume of about 200 L.
63. The method of any one of paragraphs 18 to 62, wherein the anion exchange chromatography membrane has a volume of at least about 1 mL per L of the cell culture fluid and/or the filtered cell culture fluid
64. The method of any one of paragraphs 1 to 63 additionally comprising formulating the enveloped virus into a pharmaceutical formulation or into a solution suitable for infecting a cell.
65. The method of any one of paragraphs 1 to 64, wherein the method further comprises adjusting a concentration of Mg²⁺ in the cell culture fluid or the filtered cell culture fluid to about 1-2 mM.
66. The method of paragraph 65, wherein the method further comprises adjusting a concentration of Mg²⁺ in the cell culture fluid or the filtered cell culture fluid to about 2 mM.
67. The method of any one of paragraphs 1 to 64, wherein the method further comprises adjusting the pH of the cell culture fluid or the filtered cell culture fluid to between 6.0 and 10.0.
68. The method of paragraphs 67, wherein the method further comprises adjusting the pH of the cell culture fluid or the filtered cell culture fluid to between 8.0 and 9.2.
69. The method of any one of paragraphs 1 to 64, wherein the cell culture fluid or the filtered cell culture fluid is at a temperature of 0° C. to 42° C. during contact with the endonuclease.
70. The method of paragraph 69, wherein the cell culture fluid or the filtered cell culture fluid is at a temperature of 2° C. to 8° C. during contact with the endonuclease.
71. The method of paragraph 70, wherein the cell culture fluid or the filtered cell culture fluid is at a temperature of 4° C. during contact with the endonuclease.
72. The method of any one of paragraphs 1 to 71, wherein the cell culture fluid is harvested from cells cultivated in an adherent environment.
73. The method of any one of paragraphs 1 to 71, wherein the cell culture fluid is harvested from cells cultivated in a suspension environment.
74. A purified enveloped virus produced by the method according to any one of paragraphs 1 to 73.

The present disclosure is described further in the following non-limiting examples.

### EXAMPLES

### Example 1: Methods

### Cell Culture and Lentivirus Production

Cells were grown in “iCELLis Nano” bioreactor. After four days of successful cell growth, induction of virus production began by changing the medium. Daily harvesting for up to ten days started after the discard of the first harvest volume. The harvests were collected at 4° C.

### Harvest Clarification Filtration

After the transfer of the harvest to the downstream department, a clarification filtration was performed using a Sartorius Sartopore 2 filter containing two membranes of 0.8 and 0.45 μm, respectively. The main goal of this step is to remove cells and cellular components/debris without affecting the functionality of the lentivirus or compromising its infectivity.

To study the effects of endonuclease treatment on efficiency of downstream processing Benzonase® was added to the harvest. Benzonase cuts the DNA into small fragments of 3-5 base pairs. Benzonase was added at 1 mL per liter of harvest before the clarification filtration. Addition of the endonuclease at this stage meant that the process time of the clarification filtration step was used for incubation rather than having to add additional process time.

The Benzonase working solution was prepared by diluting the stock solution 1:1000 in Dulbecco's Modified Eagle Medium (DMEM) containing 10% fetal bovine serum (FBS). For each liter of harvest, 1 mL of Benzonase working solution was added prior to the clarification filtration step.

Harvest bags and filter units were connected using tubing with an inner diameter of 8-10 mm. Before starting the actual filtration process, the membrane was equilibrated by a washing step with a volume of 0.5-0.7 mL/cm² of filter area using the equilibration buffer and drained afterwards. The equilibration as well as the subsequently performed filtration were performed at a flow rate of about 150 mL/min. The filtered harvest was then either stored at +4° C. for a maximum of 30 h or directly processed and stored at room temperature for less than one hour.

### Purification Step Using Mustang Q Anion Exchange Membrane

After filtration, virus is captured using anion exchange chromatography. The role of this capture step is to reduce the volume and to remove process-related contaminants such as host cell DNA, host cell proteins, and medium components like FBS. The chromatography step was carried out using an Äkta Pure 150 system using a Mustang Q anion exchange membrane. Table 1 shows the buffers used during anion exchange purification.

TABLE 1
Anion exchange purification parameters

| | | Flow rate in | |
|---|---|---|---|
| Step | Buffer | MV/min | MV |
| Pre-equilibration | 10 mM L-Histidine or 50 mM Tris, | Gradually | 100 |
| | 150 mM NaCl, pH 7.0 or pH 8.0 | increasing | |
| | | (2/4/6/8/10) | |
| Sanitization | 1M Sodium hydroxide | 2 | 10 |
| Pre-conditioning | 1M NaCl, 25 mM HCl | 2 | 10 |
| Equilibration | 10 mM L-Histidine, 150 mM NaCl, pH 7.0 | 10 | 5 |
| | OR | | |
| | 50 mM Tris, 150 mM NaCl, pH 8.0 | | |
| | OR | | |
| | 10 mM HEPES, 150 mM NaCl, pH 7.5 | | |
| Load | Harvest spiked with 7% 5M NaCl spike | 10 | |
| | solution | | |
| Post load wash | 10 mM L-Histidine 150 mM NaCl, pH 7.0 | 10 | 10 |
| | OR | | |
| | 50 mM Tris, 150 mM NaCl, or pH 8.0 | | |
| | OR | | |
| | 10 mM HEPES, 150 mM NaCl, pH 7.5 | | |
| Wash | 10 mM L-Histidine or 10 mM HPES or 50 | 10 | 20 |
| | mM Tris, | | |
| | 750 mM NaCl, pH 7.0 or pH 8.0 | | |
| Elution | 10 mM L-Histidine 1200 or 1500 mM | 2 | 11 |
| | NaCl, pH 7.0 | | |
| | OR | | |
| | 50 mM Tris, 1200 or 1500 mM NaCl pH | | |
| | 8.0 | | |
| | OR | | |
| | 10 mM HEPES, 1200 or 1500 mM NaCl, | | |
| | pH 7.5 | | |

Prior to the product application, the membrane was equilibrated with 5 MV of equilibration buffer at a flow rate of 10 MV/min. The filtered harvest was spiked with 5 M NaCl solution (in-line) via the built-in mixer with the help of the Äkta chromatography system to achieve a target conductivity of 40 mS/cm which reduces the non-specific binding of cell culture medium components. Separately, anion exchange chromatography was performed without NaCl spike. Harvest was then applied to the membrane at a flow rate of 10 MV/min. The membrane wash was carried out with 20 MV of wash buffer at a flow rate of 10 MV/min to remove impurities like host cell DNA. The lentivirus elution was performed with 11 MV of highly concentrated salt buffer at a flow rate of 2 MV/min. The eluate collection was started after one MV and was terminated after the 6th MV. The remaining elution volume was discarded. As the lentivirus is unstable in highly concentrated salt buffer, the eluate was directly diluted 1:10 with chilled (+4° C.) dilution buffer which is either 10 mM L-histidine or 50 mM Tris. Eluting directly into the dilution buffer reduces the time that the virus is at high salt concentration.

The collected, diluted Mustang Q eluate had a volume of 500 mL and was stored on ice for a maximum hold time of 30 minutes if the TFF step was performed as the next step directly afterwards.

### Concentration and Diafiltration Step

Tangential flow filtration (TFF) allows the concentration and diafiltration of the diluted Mustang Q eluate into the final formulation buffer X-VIVO 10. This step was performed with the Repligen KR2i system.

TFF also allowed solution exchange of the virus into the X-VIVO 10 cell culture medium to ensure that the virus can be added directly to the target cells without diluting the growth medium.

The Repligen Hollow Fibre PS membrane with a filter area of 390 cm² and 500 kDa cut-off was used.

The equilibration of the membrane was performed with 2 mL/cm² using TFF equilibration buffer. To concentrate the product, 500 mL of the diluted Mustang Q eluate in the feed reservoir were connected to the auxiliary pump. The auxiliary pump was started with a flow rate of 20 mL/min for the transfer of the feed into the reservoir. The flow rate was adjusted in such a way that the volume in the reservoir was kept constant during the concentration step. For this, the flow rate of the KR2i pump was set to 50 mL/min, the TMP to 0.5 bar (limit of 0.7 bar) and the backpressure valve was opened.

The concentration target was 25-30-fold, and the ultrafiltration step was stopped once a volume of 16-20 mL of retentate including the hold-up volume was reached. To recover the product from the retentate side of the membrane, the backpressure valve as well as the permeate line were opened. The flow rate of the main pump was set to a reverse flow of 4 mL/min to collect the hold-up recirculation volume. The retentate line which connects the backpressure valve, and the reservoir was disconnected to supply air after about one minute. This step allows to dislodge virus stuck to the membrane by applying a small inverse TMP across the membrane.

The aim of the TFF step was to achieve a concentration of 300-1000 times of the TFF retentate to that of the starting material (harvest). The TFF retentate was used for the development of the sterile filtration step.

### Sterile Filtration Studies

The sterile filtration step was carried out using the Repligen KR2i system.

### Analytics

### Infectious Titer Assay

In this assay, D1B and/or HEK293T cells are transduced with vector-containing samples. After a growth period of several days, the cells are stained with antibody against human gamma-globin to determine the infectious titer of the sample measured in transducing units (TU)/mL.

ddPCR RNA Content

This assay uses digital droplet PCR to quantify the number of RNA copies within the sample. It measures the total number of RNA copies. The primers and probes were selected to ensure that mainly full-length RNA copies are counted.

### p24 ELISA

This ELISA assay measures the viral capsid protein p24 concentration. As an additional pull-down step is established, only virus-associated and not free p24 is detected. From the readout in ng/mL an estimation of viral particles can be calculated. This assay also detects empty capsids or those with incomplete cargo RNA.

### Total DNA

This fluorescence-based assay detects all DNA within the sample.

### Example 2: Results

### Addition of Benzonase Improves Purification of Lentivirus

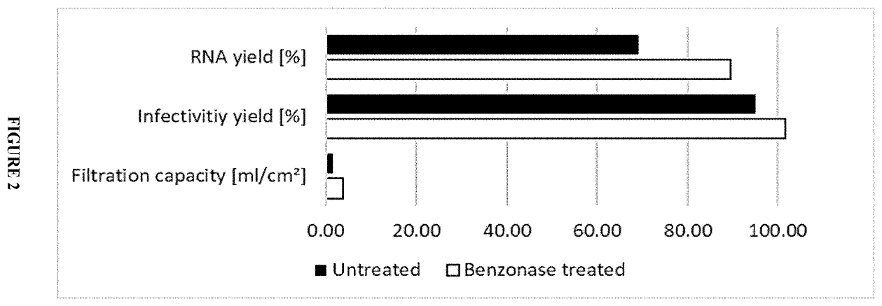

An initial feasibility study was carried out to determine if the filtration of the TFF retentate is in principle possible. Two solutions of TFF retentate, untreated and Benzonase treated, were filtered through a Mini Kleenpak EKV filter. The results are shown in FIG. 2. As shown, Benzonase treatment increased filtration capacity by about two and a half times, without affecting the infectivity yield (101.6% vs. 95.0%). In addition, the RNA yield for the Benzonase treated retentate was clearly higher with 89.5% when compared to 69.1% for the untreated retentate.

Benzonase treatment was clearly beneficial for successful filtration and was studied further.

Following the promising results in which TFF retentate was treated with Benzonase, tests were conducted in which the harvest was treated with Benzonase. Treating the harvest with Benzonase permits removal of the endonuclease by subsequent purification steps, e.g., anion exchange. To determine the success of the harvest treatment with Benzonase and to investigate the effects on the sterile filtration step, the results of this treatment were compared to the ones of the feasibility study and are presented in Table 2.

TABLE 2
Comparison of the sterile filtration results of the Benzonase
treated harvest and the Benzonase treated TFF retentate

| | Results of Benzonase | Results of Benzonase |
|---|---|---|
| Parameter | treated harvest | treated TFF retentate |
| Infectious titer yield [%] | 99.6 | 101.6 |
| RNA yield [%] | 90.1 | 89.5 |
| Filtration capacity | 2.8 | 3.7 |
| [mL/cm²] | | |

The infectivity and the RNA yield of the sterile filtration of the Benzonase-treated harvest were comparable to the results of the Benzonase-treated TFF retentate. However, the filtration capacity was decreased by 24% using the Benzonase treatment of the harvest. Despite this, all subsequent experiments used TFF retentate produced after Benzonase treatment of harvest.

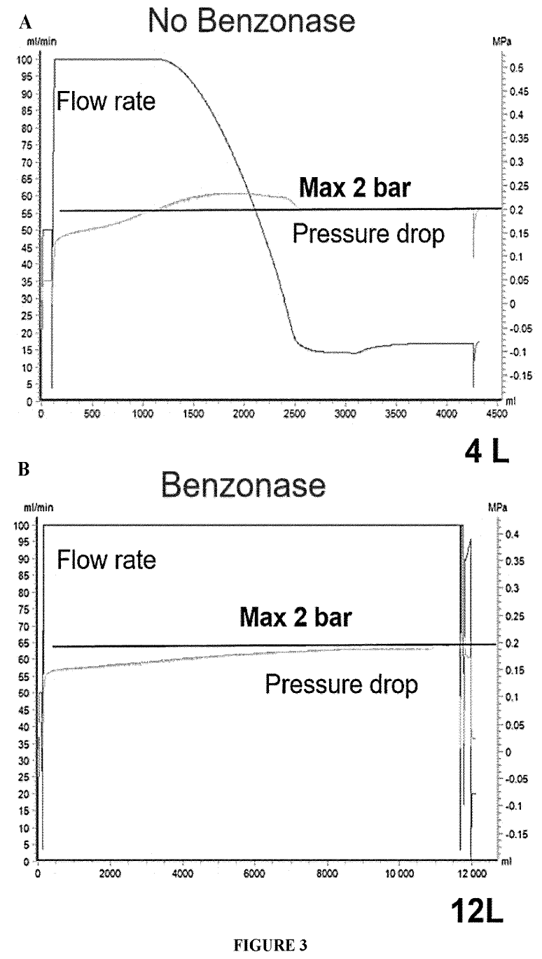

As shown in FIG. 3, addition of Benzonase to the harvest also provides positive impacts on anion exchange chromatography. In particular, without the addition of Benzonase, Mustang Q filters became fouled or blocked leading to pressure increases. In turn, this pressure increase leads to lower flow rates and increases process times (50% to 150% longer). Higher pressure also increases the risk of filter or connector failure, which can lead to a need to reprocess or discard a batch.

Thus, addition to Benzonase to the harvest improves performance of both Mustang Q filters and sterile filtration.

Improving Recovery from Anion Exchange Chromatography

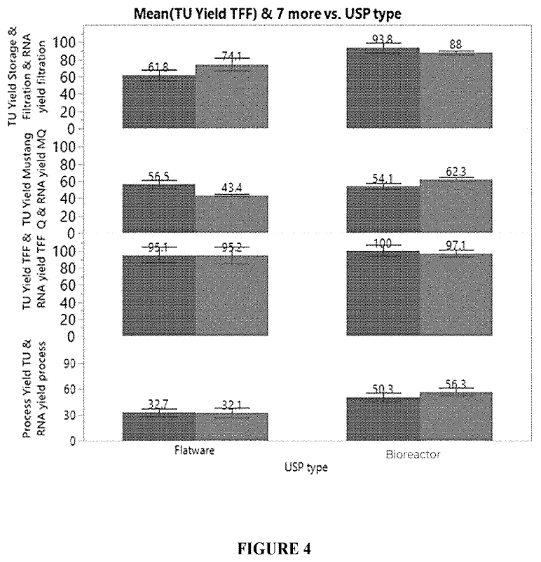

FIG. 4 is a graphical representation summarizing the yield for infectious titer (dark gray bars) and RNA content (light gray bars), for harvests collected from flatware (left side of chart) and adherent bioreactors (right side of chart). The first row shows yields from storage and filtration, the second row shows yields from the anion exchange purification step, and the third row shows yields from the TFF step. The bottom row shows the overall yields. As is shown in FIG. 4, the largest losses of virus during downstream purification occurs during anion exchange purification with 43%-63% recovery observed. Accordingly, any improvement in recovery from this step will substantially increase recovery of virus.

Several strategies were tested to determine how to improve the recovery from the anion exchange chromatography step. One of these strategies was to increase the salt concentration in the harvest. Increasing the salt concentration to about 400 mM increases the binding of the virus to the anion exchanger while reducing binding of contaminants such as host cell DNA, host cell proteins, and media components like fetal bovine serum. However, adding large volumes to the harvest is undesirable since they are difficult to mix and add to processing times and cost. On the other hand, exposing the virus to high concentrations of salt (as may occur when adding salt directly to the harvest) destabilizes the virus and results in reduced recovery. To address this issue, the inventors spiked a 5M NaCl solution directly into the anion exchange column itself rather than premixing with the virus. This reduces the contact time of the high salt concentration solution with the virus to a few seconds rather than minutes.

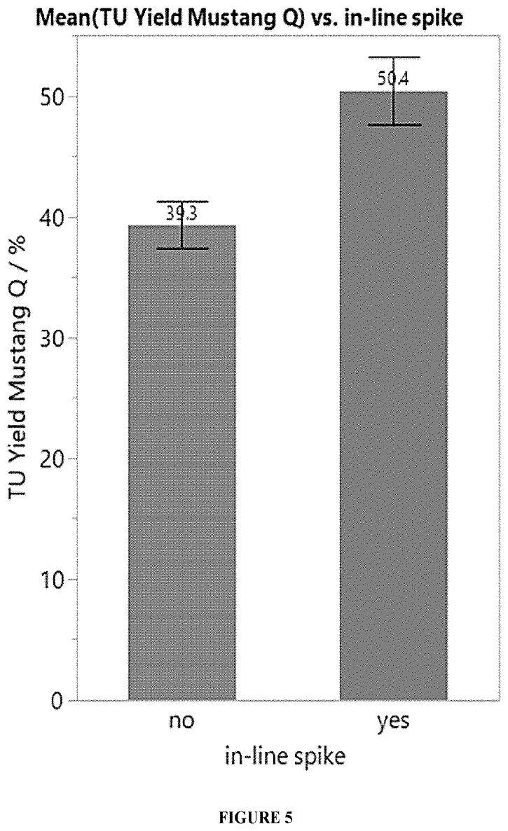

As shown in FIG. 5, the process developed by the inventors results in an improvement of approximately 10% compared to a process lacking the NaCl spike.

### Example 3: Large-Scale Sterile Filtration

To assess scale-up of the sterile filtration step, several different filters were assessed including Supor EKV (Pall, Polypropylene; 20 cm²), Sartopore Pt (Sartorius; 220 cm², 220 cm²), OptiScale Durapore (Merck, Hydrophilic PVDF; 17.8 cm²) & Sartopore Pt (Sartorius, mPES; 210 cm²).

As shown in Table 3, the Supor EKV 20 cm² sterile filtration showed>80% infectious titer yield and ˜80% RNA yield. In comparison, the OptiScale Durapore and Sartopore PT showed around 60% infectious titer yield. Further studies demonstrated that the 20 cm² filter was too small with the filtration pressure increasing quickly.

TABLE 3
Sterile filtration results using different scale-up filters

| | Optiscale | | | |
|---|---|---|---|---|
| Parameter | Durapore | Sartopore Pt | Supor EKV | |
| Size | 17.8 cm² | 210 cm² | 20 cm² | |
| Infectious titer yield | 60.6 | 60.5 | 90.9 | 81.3 |
| [%] | | | | |
| RNA yield [%] | 54.8 | 74.3 | 59 | 79.3 |
| Filtration capacity | 2.6 | (Not reached) | 4.5 | 4.5 |
| [mL/cm²] | | | | |

To address the filtration pressure issues of the Supor EKV 20 cm² filter, the larger Sartopore Pt 220 cm² filter was used. The loaded volume was 0.46 mL/cm² and the filtration capacity of the 220 cm² filter was not reached. Sterile filtration using the 220 cm² filter resulted in an average infectious titer yield of 75.4% and RNA yield of 70.0% across the 4 sub-lots tested.

Scale-up of the sterile filtration step to the 220 cm² filter was achieved in multiple runs with robust infectivity yields of approximately 80%, with all sub-lots passing safety tests with no detectable microbiological growth detected.

### Example 4: Purification of 5L Suspension Harvests

Cell culture and lentivirus production was performed as described in Example 1 however the culture was performed in a 5L suspension bioreactor. Cells were grown in a chemically defined media without fetal bovine serum (FBS).

Harvest clarification filtration was performed as described in Example 1, however the Benzonase working solution was prepared by diluting the stock solution 1:1000 in equilibration buffer (i.e., 1 μL Benzonase per 1 mL equilibration buffer) rather than FBS containing media as described in Example 1. For each liter of harvest, 1 mL of diluted Benzonase solution was added to each L of harvest prior to the clarification filtration step to achieve a target concentration of 0.3 U/mL. In addition, 10 ml of 200 mM MgCl₂ was added to achieve the target concentration of 2 mM Mg²⁺. The process parameters used for the 5L suspension run are detailed in Table 4.

TABLE 4
Process parameters for harvest clarification
filtration of the 5 L suspension run

| Parameter | Condition |
|---|---|
| Benzonase | 1 μL Benzonase in 1 mL equilibration |
| | buffer/L harvest |
| 200 mM MgCl₂ | 10 mL/1 L harvest |
| Filter | Sartorius Sartopore 2 G8 size - 1000 cm² |
| Flow rate during | 167 mL/min (=113 L/m²/h) |
| filtration (ml/min) | |

4 bioreactors were run in parallel and harvests collected and analysed on days 5 and 12. Benzonase treatment of the harvest did not affect the infectious titer yield, with an infectivity yield of 86.1% achieved in the Benzonase treated filtered harvest across 7 harvests.

After filtration, virus was captured using anion exchange chromatography with HEPES buffer similar to the method described in Example 1 above. Following addition of the Benzonase, the harvest was incubated for 50-55 min prior to loading onto the anion exchange chromatography membrane. The process parameters used for chromatography purification of the 5L suspension run are detailed in Table 5. The membrane size used is determined based on a maximum of 1000 mL harvest per 1 mL membrane volume. Accordingly, for harvests up to about 860 mL, a 0.86 mL Mustang Q membrane is used; for harvests from about 860 mL to about 1.72 L, a 1.72 mL Mustang Q membrane (or 2×0.86 mL Mustang Q membranes) are used; for harvests from about 1.72 L to about 5 L, a 5 mL Mustang Q membrane is used; for harvests from about 5L to about 10 L, a 10 mL Mustang Q membrane (or 2×5 mL Mustang Q membranes) are used; for harvests from about 10L to about 20 L, 2×10 mL Mustang Q membranes are used; for harvests from about 20 L to about 60 L, a 60 mL Mustang Q membrane is used; for harvests from about 60 L to about 120 L, a 140 mL Mustang Q membrane (or 2×60 mL Mustang Q membranes) are used; and for harvests from about 120 L to about 140 L, a 140 mL Mustang Q membrane is used.

TABLE 5
Process parameters for anion exchange chromatography

| Parameter | Condition |
|---|---|
| Membrane | 2 × 5 mL Mustang Q anion exchange |
| | membranes in parallel |
| Buffer | 10 mM HEPES, pH 7.5 (Harvest is spiked |
| | in-line with 6% 5M NaCl) |
| System | Äkta Pure 150 system |
| Dilution | Eluate: 50 mL |
| | Dilution: 450 mL buffer |

The collected, diluted Mustang Q eluate was applied to a TFF step as described in Example 1 to concentrate and diafilter the diluted Mustang Q eluate into the final formulation buffer X-VIVO 10. The process parameters used for the TFF step are described in Table 6.

TABLE 6
Process parameters for TFF

| Parameter | Condition |
|---|---|
| TFF hollow fibre | 500 kDa, 390 cm² PS Repligen membrane |
| TFF reservoir | 50 mL |
| Flow rate | 50 mL/min (=158 L/m²/h) |
| Diafiltration medium | X-Vivo 10 |
| Concentration steps | 20/25 fold (target 20 mL) |

An infectious titer yield of 46.4% of Benzonase treated TFF retentate across the 7 harvests. The results obtained by the inventors in the 5L suspension run were comparable to the results achieved in Examples 1 and 2 described above, and the infectious titer yield in FIG. 5.

### Example 5: Purification of Large Scale Harvests from Adherent Bioreactors

Cell culture and lentivirus production was performed as described in Example 1 however the culture was performed in a scale-X™ carbo bioreactor (Univercells Technologies). Harvests of 22L were collected daily for eight days. Harvest clarification filtration was performed on 44L sub-lots (every two days) with a process as described in Example 1 using the process parameters detailed in Table 7.

TABLE 7
Process parameters for harvest clarification
filtration of the large scale bioreactor

| Parameter | Condition |
|---|---|
| Benzonase | 1 μL benzonase in 1 mL equilibration |
| | buffer/L harvest |
| 200 mM MgCl₂ | 10 mL/1 L harvest |
| Filter | Sartorius Sartopore 2 G0 - 4500 cm² |
| Flow rate during filtration (ml/min) | 750 mL/min |

Anion exchange chromatography purification was also performed as described in Example 1 using HEPES buffer with in-line spiking of 6% 5M NaCl to achieve a target conductivity of 40 mS/cm. Process parameters of the chromatography purification step are provided in Table 8. The membrane size used is determined based on a maximum of 1000 mL harvest per 1 mL membrane volume, as described in Example 4, and accordingly a 60 mL Mustang Q membrane is used for the 44 L sublots.

TABLE 8
Process parameters for anion exchange chromatography

| Parameter | Condition |
|---|---|
| Membrane | 60 mL Mustang Q anion exchange membrane |
| Binding capacity | up to 1000 mL harvest/mL membrane volume |
| Max inlet pressure | 3.5 bar |
| Sanitization | 1M Sodium hydroxide |
| | Flow rate: 2 MV/min |
| Pre-conditioning | 1M NaCl, 25 mM HCl |
| | Flow rate: 2 MV/min |
| Equilibration | 10 mM HEPES, 150 mM NaCl, pH 7.5 |
| | Volume: 20 MV |
| | Flow rate: 5 MV/min |
| Load | Harvest spiked with 6% 5M NaCl spike solution |
| | Flow rate 7.5 MV/min |
| | Target conductivity: 36-44 mS/cm (at 25° C.) |
| Post load wash | 94% pump A: 10 mM HEPES, 150 mM NaCl, |
| | pH 7.5 6% pump B: 5M NaCl |
| | Volume 10 MV |
| | Flow rate: 7.5 MV/min |
| Wash | 10 mM HEPES, 750 mM NaCl, pH 7.5 |
| | Volume: 25 MV |
| | Flow rate: 7.5 MV/min |
| Elution | 10 mM HEPES, 1.5M NaCl, pH 7.5 |
| | Volume: 20 MV |
| | Flow rate: 2 MV/min |
| | Eluate collection: wait 4 MV, collect 5 MV |
| Dilution | Eluate: 300 mL |
| | Dilution: 2700 mL 10 mM Hepes, pH 7.5 |
| System | Äkta pilot 600 mL, 2-8° C. |

The collected, diluted Mustang Q eluate was applied to a TFF step as described in Example 1 to concentrate and diafilter the diluted Mustang Q eluate into the final formulation buffer X-VIVO 10. The process parameters used for the TFF step are described in Table 9.

TABLE 9
Process parameters for TFF

| Parameter | Condition | |
|---|---|---|
| TFF hollow fibre | 500 kDa, 1000 cm² | |
| TFF reservoir | 250 | mL |
| Flow rate | 250 | mL/min |

| | |
|---|---|
| Diafiltration medium | X-vivo |
| Concentration steps | 30/37.5 fold (target 20 mL) |

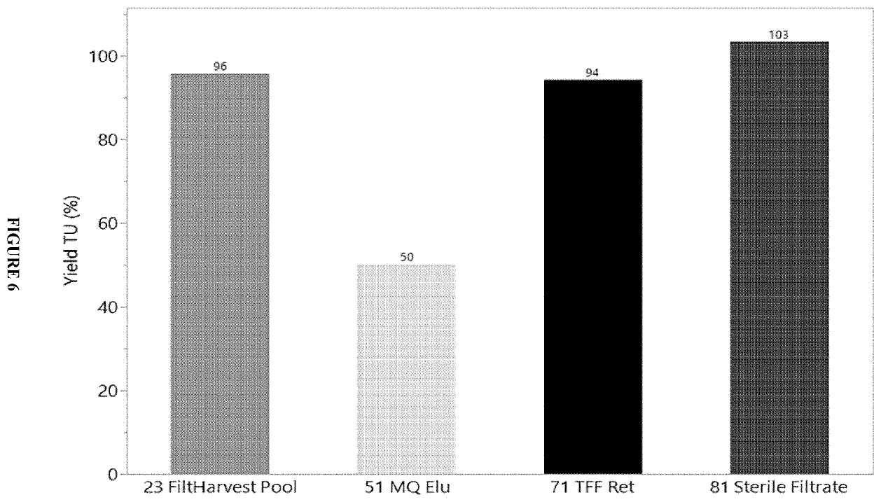

FIG. 6 shows the infectious titer yield at each process step of the processed 44L sub-lots with 96% infectious titer yield in the filtered harvest, 50% infectious titer yield in the Mustang Q eluate, 94% infectious titer yield in the TFF retentate and 103% in the sterile filtered sublot (N=4 sub-lots for each process step).

1-74. (canceled)

75. A method of purifying an enveloped virus from a cell culture fluid or a filtered cell culture fluid, comprising contacting the cell culture fluid or the filtered cell culture fluid with an endonuclease prior to purifying the virus.

76. The method of claim 75, wherein the endonuclease is contacted with the cell culture fluid or the filtered cell culture fluid at a concentration of 0.001 to 100 units/mL of cell culture fluid or filtered cell culture fluid.

77. The method of claim 75, wherein purification is performed less than about 30 hours after contacting the cell culture fluid or the filtered cell culture fluid with the endonuclease.

78. The method of claim 75, wherein the method comprises performing a harvest filtration after contacting the cell culture fluid with the endonuclease to produce the filtered cell culture fluid.

79. The method of claim 78, wherein the harvest filtration is performed immediately after contacting the cell culture fluid with the endonuclease.

80. The method of claim 75, wherein the purification comprises subjecting the filtered cell culture fluid to anion exchange chromatography.

81. The method of claim 80, wherein the filtered cell culture fluid is subjected to anion exchange chromatography immediately after harvest filtration.

82. The method of claim 81, wherein the filtered cell culture fluid is contacted with a high concentration salt solution to form a salt-spiked cell culture fluid prior to or during loading on to the anion exchange chromatography column.

83. The method of claim 80, wherein the method further comprises washing the anion exchange chromatography column with one or more wash steps.

84. The method of claim 80, wherein the method further comprises eluting bound virus from the anion exchange chromatography column with an elution solution.

85. The method of claim 84, wherein the elution solution comprises:
10-100 mM Tris, 1 M to 2 M NaCl, pH 7.0-9.0;
5-50 mM histidine, 1 M to 2 M NaCl, pH 5.5-7.4; or
5-50 mM HEPES, 1 M to 2 M NaCl, pH 6.8-8.2.

86. The method of claim 84, wherein the method further comprises diluting the eluted virus with histidine, Tris, or HEPES.

87. The method of claim 84, wherein the method further comprises concentrating and/or diafiltering the eluted virus or the diluted eluted virus.

88. A purified enveloped virus produced by the method according to claim 75.

89. A method of purifying an enveloped virus from a filtered cell culture fluid using anion exchange chromatography, wherein the filtered cell culture fluid is contacted with a high concentration salt solution to form a salt-spiked cell culture fluid prior to or during loading on to the anion exchange chromatography column.

90. The method of claim 89, wherein the filtered cell culture fluid is contacted with a high concentration salt solution to form a salt-spiked cell culture fluid immediately prior to loading on to the anion exchange chromatography column

91. The method of claim 90, wherein the high concentration salt solution and the filtered cell culture fluid are mixed in-line.

92. The method of claim 89, wherein the salt-spiked cell culture fluid has a salt concentration of between 300 and 500 mM.

93. The method of claim 89, wherein the salt-spiked cell culture fluid has a target conductivity of 35 to 45 mS/cm at 25° C.

94. A method of purifying an enveloped virus from a cell culture fluid, comprising:
(i) providing a cell culture fluid comprising viral vector produced from a stable producer cell line;
(ii) contacting the cell culture fluid with a recombinantly expressed Serratia endonuclease;
(iii) contacting the endonuclease treated cell culture fluid to a filter to produce a filtered cell culture fluid;
(iv) loading the filtered cell culture fluid and a high concentration salt solution comprising 5M sodium chloride on to an anion exchange chromatography membrane, wherein the fluid and the salt solution are loaded at a ratio of 94% (v/v) filtered cell culture fluid and 6% (v/v) high concentration salt solution;
(v) washing the membrane with one or more wash buffers;
(vi) eluting the bound virus from the membrane with an elution buffer comprising 1.2M or 1.5M sodium chloride;
(vii) diluting the eluted virus with a buffer; and
(viii) concentrating and diafiltering the eluted virus.
